(19) United States
(12) Patent Application Publication
(10) Pub. No.: US 2022/0401146 A1
(43) Pub. Date: Dec. 22, 2022

(54) TISSUE TREATMENT SYSTEMS, DEVICES, AND METHODS

(71) Applicant: Acutus Medical, Inc., Carlsbad, CA (US)

(72) Inventors: Alexander J. Asconeguy, Eagle, ID (US); Shibaji Shome, San Diego, CA (US); Ahmad Falahatpisheh, San Marcos, CA (US); Timothy J. Corvi, San Diego, CA (US); Derrick Ren-yu Chou, San Diego, CA (US); Jeffrey St. Onge, San Marcos, CA (US); Vincent J. Burgess, Encinitas, CA (US); R. Maxwell Flaherty, Topsfield, MA (US); J. Christopher Flaherty, Auburndale, FL (US)

(21) Appl. No.: 17/777,104

(22) PCT Filed: Nov. 20, 2020
(86) PCT No.: PCT/US2020/061458

§ 371 Date: May 16, 2022

Related U.S. Application Data
(60) Provisional application No. 62/939,412, filed on Nov. 22, 2019.
(60) Provisional application No. 63/075,280, filed on Sep. 7, 2020.

(51) Int. Cl. A61B 18/18; A61B 18/14; A61B 5/00

(52) U.S. Cl. CPC A61B 18/18; A61B 18/1492; A61B 5/0036; A61B 2018/00267

(57) ABSTRACT

Provided herein are systems for treating tissue of a patient. The system comprises an energy delivery console and at least one energy delivery device. The energy delivery console can provide a first dose of energy and a second dose of energy. An energy delivery device comprises a first delivery element configured to deliver the first dose of energy to target tissue, and a second delivery element configured to deliver the second dose of energy to the target tissue. The first dose of energy can comprise a delivery of energy that reversibly alters the target tissue, and the second dose of energy can comprise a delivery of energy that irreversibly alters the target tissue. The first dose of energy can be delivered to enhance a therapy provided by the second dose of energy.

### RELATED APPLICATIONS

The present application claims priority to U.S. Provisional Patent Application Ser. No. 62/939,412, entitled “Tissue Treatment Systems, Devices, and Methods”, filed Nov. 22, 2019, and U.S. Provisional Patent Application Ser. No. 63/075,280, entitled “Tissue Treatment Systems, Devices, and Methods”, filed Sep. 7, 2020, each of which is hereby incorporated by reference.

The present application, while not claiming priority to, may be related to U.S. application Ser. No. 16/335,893, entitled “Ablation System with Force Control”, filed Mar. 22, 2019, which is a 35 USC 371 national stage filing of Patent Cooperation Treaty Application No. PCT/US2017/056064, entitled “Ablation System with Force Control”, filed Oct. 11, 2017, published as WO2018/071490, which claims priority to U.S. Provisional Application Ser. No. 62/406,748, entitled “Ablation System with Force Control”, filed Oct. 11, 2016, and U.S. Provisional Application Ser. No. 62/504,139, entitled “Ablation System with Force Control”, filed May 20, 2017, each of which is hereby incorporated by reference.

The present application, while not claiming priority to, may be related to U.S. application Ser. No. 16/097,955, entitled “Cardiac Information Dynamic Display System and Method”, filed Oct. 31, 2018, which is a 35 USC 371 national stage filing of Patent Cooperation Treaty Application No. PCT/US2017/030915, entitled “Cardiac Information Dynamic Display System and Method”, filed May 3, 2017, published as WO2017/192769, which claims priority to U.S. Provisional Application Ser. No. 62/331,351, entitled “Cardiac Information Dynamic Display System and Method”, filed May 3, 2016, each of which is hereby incorporated by reference.

The present application, while not claiming priority to, may be related to U.S. patent application Ser. No. 16/861,814, entitled “Catheter, System and Methods of Medical Uses of Same, including Diagnostic and Treatment Uses for the Heart”, filed Apr. 29, 2020, which is a continuation of U.S. Pat. No. 10,667,753, entitled “Catheter, System and Methods of Medical Uses of Same, Including Diagnostic and Treatment Uses for the Heart”, filed Jun. 19, 2018, which is a continuation of U.S. Pat. No. 10,004,459, entitled “Catheter, System and Methods of Medical Uses of Same, Including Diagnostic and Treatment Uses for the Heart”, filed Feb. 20, 2015, which is a 35 USC 371 national stage filing of Patent Cooperation Treaty Application No. PCT/US2013/057579, entitled “Catheter System and Methods of Medical Uses of Same, Including Diagnostic and Treatment Uses for the Heart”, filed Aug. 30, 2013, published as WO2014/036439, which claims priority to U.S. Patent Provisional Application Ser. No. 61/695,535, entitled “System and Method for Diagnosing and Treating Heart Tissue”, filed Aug. 31, 2012, each of which is hereby incorporated by reference.

The present application, while not claiming priority to, may be related to U.S. patent application Ser. No. 16/242,810, entitled “Expandable Catheter Assembly with Flexible Printed Circuit Board (PCB) Electrical Pathways”, filed Jan. 8, 2019, which is a continuation of U.S. patent application Ser. No. 14/762,944, entitled “Expandable Catheter Assembly with Flexible Printed Circuit Board (PCB) Electrical Pathways”, filed Jul. 23, 2015, which is a 35 USC 371 national stage filing of Patent Cooperation Treaty Application No. PCT/US2014/015261, entitled “Expandable Catheter Assembly with Flexible Printed Circuit Board (PCB) Electrical Pathways”, filed Feb. 7, 2014, published as WO2014/124231, which claims priority to U.S. Patent Provisional Application Ser. No. 61/762,363, entitled “Expandable Catheter Assembly with Flexible Printed Circuit Board (PCB) Electrical Pathways”, filed Feb. 8, 2013, each of which is hereby incorporated by reference.

The present application, while not claiming priority to, may be related to U.S. patent application Ser. No. 16/533,028, entitled “Method and Device for Determining and Presenting Surface Charge and Dipole Densities on Cardiac Walls”, filed Aug. 6, 2019, which is a continuation of U.S. patent application Ser. No. 16/014,370, entitled “Method and Device for Determining and Presenting Surface Charge and Dipole Densities on Cardiac Walls”, filed Jun. 21, 2018, which is a continuation of U.S. patent application Ser. No. 15/435,763, entitled “Method and Device for Determining and Presenting Surface Charge and Dipole Densities on Cardiac Walls”, filed Feb. 17, 2017, which is a continuation of U.S. Pat. No. 9,610,024, entitled “Method and Device for Determining and Presenting Surface Charge and Dipole Densities on Cardiac Walls”, filed Sep. 25, 2015, which is a continuation of U.S. Pat. No. 9,167,982, entitled “Method and Device for Determining and Presenting Surface Charge and Dipole Densities on Cardiac Walls”, filed Nov. 19, 2014, which is a continuation of U.S. Pat. No. 8,918,158 (hereinafter the '158 patent), entitled “Method and Device for Determining and Presenting Surface Charge and Dipole Densities on Cardiac Walls”, issued Dec. 23, 2014, which is a continuation of U.S. Pat. No. 8,700,119 (hereinafter the '119 patent), entitled “Method and Device for Determining and Presenting Surface Charge and Dipole Densities on Cardiac Walls”, issued Apr. 15, 2014, which is a continuation of U.S. Pat. No. 8,417,313 (hereinafter the '313 patent), entitled “Method and Device for Determining and Presenting Surface Charge and Dipole Densities on Cardiac Walls”, issued Apr. 9, 2013, which was a 35 USC 371 national stage filing of PCT Application No. PCT/CH2007/000380, entitled “Method and Device for Determining and Presenting Surface Charge and Dipole Densities on Cardiac Walls”, filed Aug. 3, 2007, published as WO2008/014629, which claimed priority to Swiss Patent Application No. 1251/06 filed Aug. 3, 2006, each of which is hereby incorporated by reference.

The present application, while not claiming priority to, may be related to U.S. patent application Ser. No. 16/568,768, entitled “Device and Method for the Geometric Determination of Electrical Dipole Densities on the Cardiac Wall”, filed Sep. 12, 2019, which is a continuation of U.S. patent application Ser. No. 15/882,097, entitled “Device and Method for the Geometric Determination of Electrical Dipole Densities on the Cardiac Wall”, filed Jan. 29, 2018, which is a continuation of U.S. Pat. No. 9,913,589, entitled “Device and Method for the Geometric Determination of Electrical Dipole Densities on the Cardiac Wall”, filed Dec. 25, 2016, which is a continuation of U.S. Pat. No. 9,504,395, entitled “Device and Method for the Geometric Determination of Electrical Dipole Densities on the Cardiac Wall”, filed Oct. 19, 2015, which is a continuation of U.S. Pat. No. 9,192,318, entitled “Device and Method for the Geometric Determination of Electrical Dipole Densities on the Cardiac Wall”, filed Jul. 19, 2013, which is a continuation of U.S. Pat. No. 8,512,255, entitled “Device and Method for the Geometric Determination of Electrical Dipole Densities on the Cardiac Wall”, issued Aug. 20, 2013, published as US2010/0298690 (hereinafter the '690 publication), which was a 35 USC 371 national stage application of Patent Cooperation Treaty Application No. PCT/IB2009/000071 filed Jan. 16, 2009, entitled “A Device and Method for the Geometric Determination of Electrical Dipole Densities on the Cardiac Wall”, published as WO2009/090547, which claimed priority to Swiss Patent Application 00068/08 filed Jan. 17, 2008, each of which is hereby incorporated by reference.

The present application, while not claiming priority to, may be related to U.S. patent application Ser. No. 16/389,006, entitled “Device and Method for the Geometric Determination of Electrical Dipole Densities on the Cardiac Wall”, filed Apr. 19, 2019, which is a continuation of U.S. application Ser. No. 15/926,187, entitled “Device and Method for the Geometric Determination of Electrical Dipole Densities on the Cardiac Wall”, filed Mar. 20, 2018, which is a continuation of U.S. Pat. No. 9,968,268, entitled “Device and Method for the Geometric Determination of Electrical Dipole Densities on the Cardiac Wall”, filed Aug. 8, 2017, which is a continuation of U.S. Pat. No. 9,757,044, entitled “Device and Method for the Geometric Determination of Electrical Dipole Densities on the Cardiac Wall”, filed Sep. 6, 2013, which is a 35 USC 371 national stage filing of Patent Cooperation Treaty Application No. PCT/US2012/028593, entitled “Device and Method for the Geometric Determination of Electrical Dipole Densities on the Cardiac Wall”, published as WO2012/122517 (hereinafter the '517 publication), which claimed priority to U.S. Patent Provisional Application Ser. No. 61/451,357, each of which is hereby incorporated by reference.

The present application, while not claiming priority to, may be related to U.S. Design Patent Application Serial No. 29/681,827, entitled “Set of Transducer-Electrode Pairs for a Catheter”, filed Feb. 28, 2019, which is a divisional of U.S. Design Patent Application Serial No. 29/593,043, entitled “Set of Transducer-Electrode Pairs for a Catheter”, filed Feb. 6, 2017, which is a divisional of U.S. Design Pat. No. D782,686, entitled “Transducer-Electrode Pair for a Catheter”, filed Dec. 2, 2013, which is a 35 USC 371 national stage filing of Patent Cooperation Treaty Application No. PCT/US2013/057579, entitled “Catheter System and Methods of Medical Uses of Same, Including Diagnostic and Treatment Uses for the Heart”, filed Aug. 30, 2013, which claims priority to U.S. Patent Provisional Application Ser. No. 61/695,535, entitled “System and Method for Diagnosing and Treating Heart Tissue”, filed Aug. 31, 2012, each of which is hereby incorporated by reference.

The present application, while not claiming priority to, may be related to U.S. patent application Ser. No. 16/111,538, entitled “Gas-Elimination Patient Access Device”, filed Aug. 24, 2018, which is a continuation of U.S. Pat. No. 10,071,227, entitled “Gas-Elimination Patient Access Device”, filed Jul. 14, 2016, which is a 35 USC 371 national stage filing of Patent Cooperation Treaty Application No. PCT/US2015/11312, entitled “Gas-Elimination Patient Access Device”, filed Jan. 14, 2015, which claims priority to U.S. Patent Provisional Application Ser. No. 61/928,704, entitled “Gas-Elimination Patient Access Device”, filed Jan. 17, 2014, which is hereby incorporated by reference.

The present application, while not claiming priority to, may be related to U.S. patent application Ser. No. 15/128,563, entitled “Cardiac Analysis User Interface System and Method”, filed Sep. 23, 2016, which is a 35 USC 371 national stage filing of Patent Cooperation Treaty Application No. PCT/US2015/22187, entitled “Cardiac Analysis User Interface System and Method”, filed Mar. 24, 2015, which claims priority to U.S. Patent Provisional Application Ser. No. 61/970,027, entitled “Cardiac Analysis User Interface System and Method”, filed Mar. 28, 2014, which is hereby incorporated by reference.

The present application, while not claiming priority to, may be related to U.S. patent application Ser. No. 17/063,901, entitled “Devices and Methods for Determination of Electrical Dipole Densities on a Cardiac Surface”, filed Oct. 6, 2020, which is a continuation of U.S. Pat. No. 10,828,011, entitled “Devices and Methods for Determination of Electrical Dipole Densities on a Cardiac Surface”, filed Mar. 2, 2016, which is a 35 USC 371 national stage filing of Patent Cooperation Treaty Application No. PCT/US2014/54942, entitled “Devices and Methods for Determination of Electrical Dipole Densities on a Cardiac Surface”, filed Sep. 10, 2014, which claims priority to U.S. Patent Provisional Application Ser. No. 61/877,617, entitled “Devices and Methods for Determination of Electrical Dipole Densities on a Cardiac Surface”, filed Sep. 13, 2013, which is hereby incorporated by reference.

The present application, while not claiming priority to, may be related to U.S. patent application Ser. No. 16/849,045, entitled “Localization System and Method Useful in the Acquisition and Analysis of Cardiac Information”, filed Apr. 15, 2020, which is a continuation of U.S. Pat. No. 10,653,318, entitled “Localization System and Method Useful in the Acquisition and Analysis of Cardiac Information”, filed Oct. 26, 2017, which is a 35 USC 371 national stage filing of Patent Cooperation Treaty Application No. PCT/US2016/032420, entitled “Localization System and Method Useful in the Acquisition and Analysis of Cardiac Information”, filed May 13, 2016, which claims priority to U.S. Patent Provisional Application Ser. No. 62/161,213, entitled “Localization System and Method Useful in the Acquisition and Analysis of Cardiac Information”, filed May 13, 2015, which is hereby incorporated by reference.

The present application, while not claiming priority to, may be related to U.S. patent application Ser. No. 15/569,231, entitled “Cardiac Virtualization Test Tank and Testing System and Method”, filed Oct. 25, 2017, which is a 35 USC 371 national stage filing of Patent Cooperation Treaty Application No. PCT/US2016/031823, filed May 11, 2016, which claims priority to U.S. Patent Provisional Application Ser. No. 62/160,501, entitled “Cardiac Virtualization Test Tank and Testing System and Method”, filed May 12, 2015, which is hereby incorporated by reference.

The present application, while not claiming priority to, may be related to U.S. patent application Ser. No. 15/569,185, entitled “Cardiac Virtualization Test Tank and Testing System and Method”, filed Oct. 25, 2017, which is a 35 USC 371 national stage filing of Patent Cooperation Treaty Application No. PCT/US2016/032017, filed May 12, 2016, which claims priority to U.S. Patent Provisional Application Ser. No. 62/160,529, entitled “Ultrasound Sequencing System and Method”, filed May 12, 2015, which is hereby incorporated by reference.

The present application, while not claiming priority to, may be related to U.S. patent application Ser. No. 16/097,959, entitled “Cardiac Mapping System with Efficiency Algorithm”, filed Oct. 31, 2018, which is a 35 USC 371 national stage filing of Patent Cooperation Treaty Application No. PCT/US2017/030922, entitled “Cardiac Mapping System with Efficiency Algorithm”, filed May 3, 2017, which claims priority to U.S. Patent Provisional Application Ser. No. 62/413,104, entitled “Cardiac Mapping System with Efficiency Algorithm”, filed Oct. 26, 2016, which is hereby incorporated by reference.

The present application, while not claiming priority to, may be related to U.S. patent application Ser. No. 16/961,809, entitled “System for Identifying Cardiac Conduction Patterns”, filed Jul. 13, 2020, which is a 35 USC 371 national stage filing of Patent Cooperation Treaty Application No. PCT/US2019/014498, entitled “System for Identifying Cardiac Conduction Patterns”, filed Jan. 22, 2019, which claims priority to U.S. Patent Provisional Application Ser. No. 62/619,897, entitled “System for Recognizing Cardiac Conduction Patterns”, filed Jan. 21, 2018, and U.S. Patent Provisional Application Ser. No. 62/668,647, entitled “System for Identifying Cardiac Conduction Patterns”, filed May 8, 2018, each of which is hereby incorporated by reference.

The present application, while not claiming priority to, may be related to U.S. patent application Ser. No. 17/048,151, entitled “Cardiac Information Processing System”, filed Oct. 16, 2020, which is a 35 USC 371 national stage filing of Patent Cooperation Treaty Application No. PCT/US2019/031131, entitled “Cardiac Information Processing System”, filed May 7, 2019, which claims priority to U.S. Provisional Application Ser. No. 62/668,659, entitled “Cardiac Information Processing System”, filed May 8, 2018, and U.S. Patent Provisional Application Ser. No. 62/811,735, entitled “Cardiac Information Processing System”, filed Feb. 28, 2019, each of which is hereby incorporated by reference.

The present application, while not claiming priority to, may be related to Patent Cooperation Treaty Application No. PCT/US2019/060433, entitled “Systems and Methods for Calculating Patient Information”, filed Nov. 8, 2019, which claims priority to U.S. Provisional Application Ser. No. 62/757,961, entitled “Systems and Methods for Calculating Patient Information”, filed Nov. 9, 2018, each of which is hereby incorporated by reference.

The present application, while not claiming priority to, may be related to Patent Cooperation Treaty Application No. PCT/US2020/028779, entitled “System for Creating a Composite Map”, filed Apr. 17, 2020, which claims priority to U.S. Provisional Application Ser. No. 62/835,538, entitled “System for Creating a Composite Map”, filed Apr. 18, 2019, and U.S. Provisional Application Ser. No. 62/925,030, entitled “System for Creating a Composite Map”, filed Oct. 23, 2019, each of which is hereby incorporated by reference.

The present application, while not claiming priority to, may be related to Patent Cooperation Treaty Application No. PCT/US2020/036110, entitled “Systems and Methods for Performing Localization Within a Body”, filed Jun. 4, 2020, which claims priority to U.S. Provisional Application Ser. No. 62/857,055, entitled “Systems and Methods for Performing Localization Within a Body”, filed Jun. 4, 2019, each of which is hereby incorporated by reference.

### FIELD OF THE INVENTION

The present inventive concepts relate generally to systems, devices, and methods for ablating tissue, and in particular, for ablating tissue of a patient's heart.

### BACKGROUND

Numerous medical procedures include the delivery of energy to ablate or otherwise treat tissue. Achieving desired specificity and efficacy of tissue treatment can be challenging, and it can result in less than desired results.

There is a need for systems, methods, and devices that achieve improved tissue treatment via delivery of energy.

### SUMMARY

According to an aspect of the present inventive concepts, a system for treating tissue of a patient, the system comprises: an energy delivery console for providing a first dose of energy and a second dose of energy; and an energy delivery device comprising a first delivery element configured to deliver the first dose of energy to target tissue, and a second delivery element configured to deliver the second dose of energy to the target tissue. The first dose of energy can comprise a delivery of energy that reversibly alters the target tissue. The second dose of energy can comprise a delivery of energy that irreversibly alters the target tissue. The first dose of energy can be delivered to enhance a therapy provided by the second dose of energy.

In some embodiments, the target tissue comprises cardiac tissue.

In some embodiments, the target tissue comprises nerve tissue.

In some embodiments, the target tissue comprises vessel wall tissue.

In some embodiments, the target tissue comprises tissue of an organ.

In some embodiments, the target tissue comprises tissue selected from the group consisting of: cardiac tissue; nerve tissue; vessel wall tissue; organ tissue; brain tissue; lung tissue; kidney tissue; liver tissue; stomach tissue; muscle tissue; and combinations thereof.

In some embodiments, the energy delivery device comprises a catheter.

In some embodiments, the energy delivery device comprises a device selected from the group consisting of: catheter; surgical tool; laparoscopic tool; endoscopic tool; and combinations thereof.

In some embodiments, the first energy delivery element and the second energy delivery element comprise the same component.

In some embodiments, the first energy delivery element and the second energy delivery element comprise different components.

In some embodiments, the first energy delivery element comprises multiple energy delivery elements. The second energy delivery element can comprise a single energy delivery element. The second energy delivery elements can comprise multiple energy delivery elements that are the same components as the first energy delivery elements.

In some embodiments, the second energy delivery element comprises multiple energy delivery elements. The first energy delivery element can comprise a single energy delivery element.

In some embodiments, the energy delivery device comprises a first energy delivery device and a second energy delivery device, and the multiple energy delivery elements comprises a first device element of the first energy delivery device and a second device element of the second energy delivery device. During the delivery of the second dose, the first device element can be configured to be positioned on the endocardial surface of the patient's heart and the second device element can be configured to be positioned on the epicardial surface of the patient's heart.

In some embodiments, the first dose of energy comprises a delivery of energy insufficient to ablate, cause necrosis of, and/or otherwise permanently alter the target tissue, and the second dose of energy comprises a delivery of energy sufficient to ablate, cause necrosis of, and/or otherwise permanently alter the target tissue. In some embodiments, parameters of the first dose of energy and/or the second dose of energy are determined by an algorithm of the system, such as an artificial intelligence-based algorithm.

In some embodiments, the system is configured to deliver the first dose of energy and/or the second dose of energy to an endocardial tissue surface.

In some embodiments, the system is configured to deliver the first dose of energy and/or the second dose of energy to an epicardial tissue surface.

In some embodiments, the system is configured to deliver the second dose of energy after the first dose of energy has been delivered. The second dose of energy can be configured to irreversibly electroporate the target tissue.

In some embodiments, the system is configured to deliver the second dose of energy during at least a portion of the delivery of the first dose of energy.

In some embodiments, the first dose of energy comprises RF energy delivered at a level insufficient to ablate tissue.

In some embodiments, the first dose of energy comprises a form of energy selected from the group consisting of: thermal energy; heat energy; cryogenic energy; electromagnetic energy; radiofrequency (RF) energy; microwave energy; light energy; laser light energy; sound energy; subsonic energy; ultrasonic energy; chemical energy; and combinations thereof.

In some embodiments, the second dose of energy comprises a form of energy selected from the group consisting of: thermal energy; heat energy; cryogenic energy; electromagnetic energy; radiofrequency (RF) energy; microwave energy; light energy; laser light energy; sound energy; subsonic energy; ultrasonic energy; chemical energy; and combinations thereof.

In some embodiments, the first dose of energy and the second dose of energy comprise different forms of energy.

In some embodiments, the second dose of energy comprises an irreversible electroporation pulse of energy. The second dose can comprise a parameter selected from the group consisting of: a dose delivered by an electrode with a length of at least 1.46 mm, and/or a length of no more than 8 mm; a dose delivered by a pair of electrodes that are separated by at least 1 mm, and/or separated by no more than 11 mm; a dose based on a provided voltage of at least 500V and/or no more than 5000V; a dose comprising an electric field strength of at least 200V/cm and/or no more than 1000V/cm; a dose comprising a pulse width of at least 0.1 μsec and/or no more than 200 μsec; a dose comprising a series of pulses with a pulse repetition interval of at least 1 μsec; and combinations thereof.

In some embodiments, the first dose of energy is delivered for a fixed time period. The system can be configured to monitor the heart cycle of the patient, and the second dose of energy can be initiated when the heart cycle reaches a desired heart cycle point. The system can be configured to enter an alert mode if the first dose is delivered and a timeout period is reached prior to the second dose being delivered.

In some embodiments, the system is configured to monitor the patient's heart cycle during the delivery of the first dose and/or during the delivery of the second dose. The system can be configured to monitor the patient's heart cycle during both the delivery of the first dose and the delivery of the second dose. The first dose can be delivered until the patient's heart cycle reaches a desired heart cycle point or until a timeout is reached.

In some embodiments, the system is configured to monitor the patient's heart prior to the delivery of the first dose of energy. The system can be configured to predict a time T1 of the next desired heart cycle point after receiving an energy delivery signal. The first dose of energy can comprise energy delivery parameters based on a target amount of energy to be delivered and a time period to reach T1. The second dose of energy can be delivered if the patient's heart cycle is determined by the system to be equal to the desired heart cycle point at time T1. The second dose of energy may not be delivered if the patient's heart cycle is determined to be different than the desired heart cycle point at time T1.

In some embodiments, the first dose of energy is configured to cause at least a 2° C. increase in temperature of the target tissue.

In some embodiments, the system is configured to deliver a third dose of energy and a fourth dose of energy to additional target tissue, and: the first dose of energy comprises a delivery of energy that reversibly alters the target tissue; and the second dose of energy comprises a delivery of energy that irreversibly alters the target tissue. The third dose of energy can be similar to the first dose of energy, and the fourth dose of energy can be similar to the second dose of energy.

In some embodiments, the system further comprises a monitoring device configured to provide physiologic information of the patient, and the energy delivery console provides the first dose of energy and/or the second dose of energy based on the physiologic information provided. The physiologic information can comprise cardiac cycle information. The physiologic information can comprise information related to a physiologic parameter selected from the group consisting of: cardiac cycle; heart rate; blood pressure; blood flow rate; respiration rate; brain activity; electrogram amplitude; tissue impedance; and combinations thereof.

According to another aspect of the present inventive concepts, a method for delivering energy to cardiac tissue, comprises: (1) inserting a device comprising at least one electrical energy delivery element into the heart chamber of a patient; (2) positioning the at least one electrical energy delivery element proximate a target location comprising target tissue to receive energy; and (4) delivering a dose of energy to the target tissue, the dose of energy sufficient to irreversibly electroporate the target tissue. The method can further comprise predicting a time T1 of a subsequent desired heart cycle point, wherein step (4) is performed at time T1. The method can further comprise performing the following prior to step (4): (3) delivering an additional dose of energy that causes an increase in the temperature of the target tissue. The additional dose of energy can comprise a delivery of RF energy. The RF energy can be delivered for a fixed time period. Step (4) may not be performed at time T1 if the patient's heart cycle is not equal to the desired heart cycle point.

According to another aspect of the present inventive concepts, a method for delivering energy to cardiac tissue, comprises: (1) inserting a device into a heart chamber of a patient, the device comprising at least one electrical energy delivery element; (2) positioning the at least one electrical energy delivery element proximate a target location comprising target tissue to receive energy; (3) heating the target tissue; and (4) subsequently delivering a second energy to the target tissue, the second energy configured to irreversibly electroporate the target tissue.

According to another aspect of the present inventive concepts, a system for treating tissue of a patient, the system comprises: an energy delivery console for providing an electrical pulse configured to perform pulsed field ablation of target tissue; an energy delivery device comprising two or more electrodes; and a graphical user interface. The electrical pulse is delivered between the two or more electrodes, and the graphical user interface is configured to provide information relating to the strength of the electric field generated by the electric pulse.

According to another aspect of the present inventive concepts, a system for treating tissue of a patient, the system comprises: an energy delivery console for providing an electrical pulse configured to perform pulsed field ablation of target tissue; an energy delivery device comprising two or more electrodes; and irrigation fluid comprising a different conductance than the conductance of blood. The electrical pulse is delivered between the two or more electrodes, and the system is configured to steer the electric field generated by the electric pulse via delivery of the irrigation fluid.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a schematic view of a system for performing a medical procedure on a patient, consistent with the present inventive concepts.

FIG. 2 illustrates a flow chart of a method for delivering energy to tissue of a patient, consistent with the present inventive concepts.

FIG. 3 illustrates a flow chart of another method for delivering energy to tissue of a patient, consistent with the present inventive concepts.

FIG. 3A illustrates a graph of a heart cycle including a desired cycle point, consistent with the present inventive concepts.

FIG. 4 illustrates a flow chart of another method for delivering energy to tissue of a patient, consistent with the present inventive concepts.

FIG. 5 illustrates a flow chart of another method for delivering energy to tissue of a patient, consistent with the present inventive concepts.

FIG. 6 illustrates a flow chart of another method for delivering energy to tissue of a patient, consistent with the present inventive concepts.

FIG. 7 illustrates a flow chart of another method for delivering energy to tissue of a patient, consistent with the present inventive concepts.

FIG. 8 illustrates a side view of an energy delivery device, consistent with the present inventive concepts.

FIGS. 8A-B are two graphs illustrating lesion depths created with various energy delivery geometries, consistent with the present inventive concepts.

FIGS. 9A-D are four graphs illustrating lesion volumes created with various energy delivery geometries, consistent with the present inventive concepts.

FIGS. 10A-B are two anatomical sectional views of the distal portion of an energy delivery device contacting a tissue surface at different angles of orientation, consistent with the present inventive concepts.

FIGS. 11A-B are two user's views of a graphical user interface displaying information related to different angles of orientation of an energy delivery device, consistent with the present inventive concepts.

FIG. 12 is a perspective view of the distal portion of an energy delivery device including multiple ports for delivering irrigation fluid, consistent with the present inventive concepts.

FIG. 13 is a side sectional anatomical view of the distal portion of an energy delivery device in contact with a tissue surface and delivering irrigation fluid, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are systems, devices, and methods for treating tissue of a patient. An energy delivery console can be configured to deliver various “doses” of energy to be delivered by one or more energy delivery devices such as catheters or surgical tools that include electrodes or other energy delivery elements. In some embodiments, multiple, inter-dependent energy doses are delivered to a common tissue location, such as to provide an improved therapeutic benefit to the patient. An initial dose can be configured to warm tissue, such as a delivery of radiofrequency (RF), heat, and/or other energy. A subsequent dose can comprise a dose of energy configured to irreversibly electroporate the tissue that had previously been warmed, such as while that tissue is in an elevated temperature (e.g. above body temperature) state.

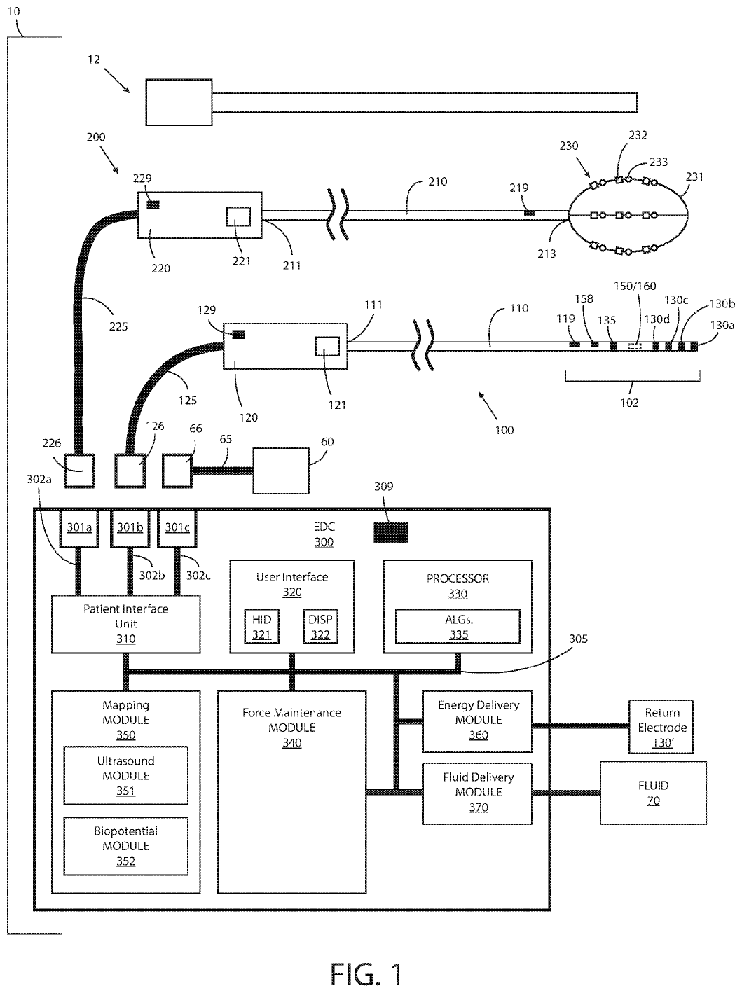

Referring now to FIG. 1, a schematic view of a system for performing a medical procedure on a patient (e.g. a human or other living mammal) is illustrated, consistent with the present inventive concepts. The medical procedure can comprise a diagnostic procedure, a therapeutic procedure, or a combined diagnostic and therapeutic procedure. System 10 can comprise one or more ablation catheters and/or other energy delivery devices, EDD 100, one or more mapping devices, mapping catheter 200, one or more sheaths, sheath 12, one or more patient patches, patch 60, and/or a console for delivering energy, EDC 300. EDC 300 operably attaches (e.g. electrically, mechanically, fluidly, sonically, and/or optically attaches) to the one or more devices 100, 200 (e.g., two, three or more devices 100, 200), and/or to the one or more patient patches 60.

EDC 300 can comprise a console or other device configured to deliver one or more forms of energy (e.g. deliver energy to tissue via a catheter or other energy delivery device of system 10). As used herein, delivery of energy to tissue shall include the transfer of energy to tissue (e.g. to heat, ablate, and/or otherwise affect tissue) as well as the extraction of energy from tissue (e.g. to cool, freeze, and/or cryogenically ablate tissue). EDC 300 can be configured to deliver energy to tissue (e.g. via EDD 100) to create a lesion in tissue, such as to create one or more therapeutic lesions in heart tissue to treat atrial fibrillation (AF), and/or other arrhythmia of the patient.

EDC 300 can deliver the one or more forms of energy to one or more electrodes and/or other energy delivery elements 130 of EDD 100 (also referred to as electrodes 130 herein). In FIG. 1, EDD 100 comprises four energy delivery elements 130, an element at the distal end of EDD 100 (e.g. a “tip electrode”), and three elements 130 mounted more proximally, elements 130b-d (e.g. “ring electrodes”). In some embodiments, EDD 100 comprises between 1 and 64 energy delivery elements 130, such as between 1 and 12 elements 130 positioned in a linear or curvilinear arrangement.

In some embodiments, EDC 300 can be configured to deliver a first dose of energy, dose DOE1, and a second dose of energy, dose DOE2, where dose DOE2 is different than dose DOE1 (e.g. doses DOE1 and DOE2 comprise different types of energy, levels of energy, waveforms of energy delivery, durations of energy delivery, and/or other differing energy parameter). In some embodiments, doses DOE1 and DOE2 have multiple differences in energy delivery parameters between them. In some embodiments, dose DOE1 is delivered to a first portion of tissue, and dose DOE2 is delivered to a second portion of tissue. The first and second portions of tissue can be the same portion of tissue. At least a portion of the first portion of tissue can be included in the second portion of tissue.

Dose DOE1 can comprise a delivery of energy that reversibly alters target tissue (e.g. a volume of tissue intended to be reversibly altered by dose DOE1), while dose DOE2 can comprise a delivery of energy that irreversibly alters the target tissue (e.g. a volume of tissue intended to be irreversibly altered by dose DOE2). Changes, or lack of changes, to tissue (e.g. target tissue) shall be described herein in terms of the effects encountered by a majority, but not necessarily all, of the target tissue. For example, as used herein, “reversibly altering tissue”, and the like, can refer to a reversible altering of all, or simply a majority of tissue (e.g. a majority of target tissue), in other words, a small portion (e.g. less than 30%, 20%, or 10%) of the target tissue (e.g. tissue intended to be reversibly altered by dose DOE1) may be irreversibly altered by the delivery of the energy, or not altered at all, while the majority of the target tissue (e.g. at least 70%, 80%, or 90%, respectively) is reversibly altered. Similarly, as used herein, “irreversibly altering tissue”, and the like, can refer to an irreversible altering of all, or simply a majority of target tissue (e.g. a majority of tissue intended to be altered by dose DOE2), in other words, a small portion (e.g. less than 30%, 20%, or 10%) of the target tissue may be reversibly altered by the delivery of the energy, or not altered at all, while the majority of the target tissue (e.g. at least 70%, 80%, or 90%, respectively) is irreversibly altered.

Dose DOE1 can be configured to enhance the effect (e.g. the tissue effect) caused by dose DOE2 (e.g. when at least a portion of dose DOE2 is delivered after completion of the delivery of dose DOE1), such as is described herein.

Dose DOE1 can comprise a delivery of energy that is below a threshold, such as a threshold of delivered energy that causes the target tissue (e.g. the tissue receiving the energy and potentially some neighboring tissue) to change from an initial state (e.g. an initial temperature, pressure, level of cell membrane permeability, viability level, state of health, and/or other tissue state), and then subsequently return to that initial state over time (e.g. within 10 minutes, 1 hour, or 1 day). For example, dose DOE1 can comprise a delivery of energy that simply causes the cooling or warming of target tissue from body temperature, where the target tissue returns to body temperature within a relatively short time period after the cessation of energy delivery, such as when dose DOE1 comprises an energy delivery that is insufficient to ablate, insufficient to cause necrosis of, and/or otherwise insufficient to permanently alter the target tissue (e.g. an RF energy delivery that has an amplitude, frequency, duration, and/or other parameter that is insufficient to ablate, necrose, and/or otherwise permanently alter the target tissue). Dose DOE1 can comprise a delivery of RF energy that is delivered in a monopolar mode (e.g. between energy delivery element 130a and/or other energy delivery elements 130 of EDD 100 and return electrode 130′), and/or RF energy that is delivered in a bipolar mode (e.g. between two delivery elements 130 of EDD 100). Dose DOE1 can comprise a delivery of non-electrical energy (e.g. light energy, ultrasound energy, and/or thermal energy) that causes an increase in the temperature of the tissue receiving dose DOE1 (e.g. the same tissue to receive dose DOE2).

Dose DOE2 can comprise a delivery of energy that is above a threshold, such as a threshold of delivered energy that causes the target tissue (e.g. the tissue receiving the energy and potentially some neighboring tissue) to change from an initial state (e.g. an initial pressure, level of cell membrane permeability, viability level, state of health, and/or other tissue state) without subsequently returning to that initial state over time (e.g. not within a time period of 4 hours, 1 week, 1 month, 3 months, 6 months, 1 year, or 2 years). For example, dose DOE2 can comprise a delivery of energy that causes an irreversible change and/or other desired long-term effect to target tissue, such as when dose DOE2 comprises an energy level that is sufficient to ablate, sufficient to cause necrosis of, and/or otherwise sufficient to permanently alter the target tissue (e.g. energy delivered in the form of an IEP dose, described herebelow, that has an amplitude, frequency, duration, and/or other parameter that is sufficient to create a desired lesion in tissue, such as to treat AF or other arrhythmia of the patient).

Doses DOE1 and DOE2 can be delivered sequentially, such as when dose DOE2 is delivered immediately after or at least soon after the completion of the delivery of dose DOE1. In some embodiments, at least a portion of the delivery of dose DOE2 (e.g. an initial portion of dose DOE2) is delivered during at least a portion of the delivery of dose DOE1 (e.g. final portion of dose DOE1), such as in an overlapping and/or interleaving arrangement.

In some embodiments, dose DOE2 comprises a delivery of energy that causes irreversible electroporation of the target tissue. For example, dose DOE2 can comprise the delivery of an “IEP” dose. An IEP dose, as used herein, can comprise one or more electrical pulses delivered between two or more electrodes, the pulses configured to generate an electric field within tissue proximate the two electrodes. The parameters of the electrical pulses can be selected such that the resultant electric field causes the irreversible electroporation of the tissue, such as when significant thermal damage to the tissue is avoided (e.g. delivery of excessive heat to tissue is avoided). For example, the IEP dose can be configured to prevent the tissue receiving the dose from exceeding a temperature of 50° C. In some embodiments, the IEP dose is configured to limit a resultant temperature increase in tissue (e.g. the tissue receiving the IEP dose) to a temperature increase that does not exceed 13° C., 11° C., 9° C., or 7° C.

In some embodiments, the IEP dose is delivered by an EDD 100 with an electrode-based energy delivery element 130 with a length of at least 1.46 mm, and/or a length of no more than 8 mm. In some embodiments, the IEP dose is delivered by an EDD 100 with two electrode-based energy delivery elements 130 that are separated by at least 1 mm, and/or separated by no more than 11 mm. In some embodiments, the IEP dose is delivered based on a provided voltage (e.g. provided by EDC 300) of at least 500V, and/or no more than 5000V. In some embodiments, the IEP dose comprises a field strength of at least 200V/cm, and/or no more than 1000V/cm. In some embodiments, the IEP dose comprises a pulse width of at least 0.1 μsec, and/or no more than 200 μsec. In some embodiments, the IEP dose comprises a series of pulses with a pulse repetition interval of at least 1 μsec.

In some embodiments, an IEP dose of the present inventive concepts comprises an energy delivery parameter level selected from the group consisting of: a voltage gradient of at least 50V/cm, at least 100V/cm; at least 300V/cm, or at least 400V/cm; a voltage gradient of no more than 8000V/cm, or no more than 800V/cm; an amplitude of no more than 5000V; an amplitude of no more than 2000V; an amplitude of no more than 1000V; a set of at least 2 pulses; a set of no more than 15 pulses; a set of pulses, each of at least 1 microsecond in duration; an IEP duration of at least 5 microseconds; an IEP duration of no more than 30 seconds; and combinations thereof. In some embodiments, dose DOE2 comprises an IEP dose that is delivered between two electrode-based energy delivery elements 130 that are positioned at least 2 mm, 5 mm, 7 mm, or 10 mm apart. In some embodiments, one of the delivery elements 130 receiving and/or delivering the IEP dose is positioned at the distal end (tip) of EDD 100 (e.g. element 130a shown). In some embodiments, one or both of the delivery elements 130 receiving and/or delivering the IEP dose comprises an electrode that is circular in shape (e.g. a ring electrode).

In some embodiments, dose DOE2 comprises an IEP dose, and dose DOE1 comprises a delivery of energy (e.g. RF energy) that warms the target tissue (e.g. tissue warming that is performed prior to delivery of dose DOE2). The warming caused by dose DOE1 can provide one or more benefits, such as: a reduction in the required amplitude of the IEP dose of dose DOE2 to achieve successful energy delivery (achieve a successful lesion creation); a decrease in the duration of the IEP dose; a modification of the frequency of the IEP dose; a modification of the waveform shape of the IEP dose; and/or improve the efficacy (ablative effects) of the IEP dose. In some embodiments, dose DOE1 is configured to cause the tissue receiving the dose to increase at least 2° C., such as at least 3° C. or at least 4° C.

In some embodiments, dose DOE2 comprises an IEP dose that is delivered by one or more pairs of electrode-based energy delivery elements 130, such as when one of the elements 130 of each pair is configured as a cathode, and the other is configured as an anode. System 10 (e.g. EDC 300) can be configured (e.g. via algorithm 335 described herein) to select which elements 130 are to deliver the IEP dose (e.g. which pair of a set of three or more elements 130), as well as which element 130 is to be the cathode and which is to be the anode. In some embodiments, a tip-positioned element 130 (e.g. element 130a of FIG. 1, positioned on the distal end of shaft 110) is configured as the cathode, and a more proximal element 130 (e.g. a ring electrode, such as one or more of the elements 130b-d of FIG. 1) is configured as the anode. In some embodiments, dose DOE1 and/or DOE2 comprises delivery of electrical energy between a pair of electrodes comprising one or more elements 130 configured as an anode, and one or more elements 130 configured as a cathode. For example, system 10 (e.g. one or more EDD's 100) can comprise multiple energy delivery elements 130 configured to function as an anode, a cathode, or both.

Dose DOE2 can comprise an IEP dose that is delivered while the impedance of the tissue receiving the dose is monitored by system 10, such as when system 10 delivers the IEP dose in a closed-loop fashion, and/or when successful irreversible electroporation of the tissue is confirmed via the impedance measurement (e.g. and system 10 automatically stops the IEP dose upon the confirmation).

Doses DOE1 and/or DOE2 can be delivered by one or more energy delivery elements 130 to one or more types of target tissue, such as cardiac tissue, nerve tissue, vessel wall tissue, and/or organ tissue. In some embodiments, doses DOE1 and/or DOE2 can be configured to be delivered to tissue selected from the group consisting of: cardiac tissue; nerve tissue; vessel wall tissue; organ tissue; brain tissue; lung tissue; kidney tissue; liver tissue; stomach tissue; muscle tissue; and combinations thereof. Doses DOE1 and/or DOE2 can be delivered to the surface of an organ (e.g. an endocardial and/or epicardial surface of the heart), and/or within tissue of an organ (e.g. within heart wall tissue and/or within another type of organ tissue).

EDC 300 can comprise an energy delivery module, module 360 shown, such as an energy delivery module configured to provide ablation energy to EDD 100 (e.g. provide energy of doses DOE1 and DOE2 and/or other energy to one or more energy delivery elements 130 comprising one or more electrodes and/or other energy delivery elements). Energy delivery module 360 can provide energy to EDD 100 via a patient interface unit, PIU 310 (as shown and described herein), or otherwise. As described herein, energy provided by module 360 can comprise an energy form selected from the group consisting of: thermal energy, such as heat energy or cryogenic energy; electromagnetic energy, such as radiofrequency (RF) energy and/or microwave energy; light energy, such as light energy provided by a laser; sound energy, such as subsonic energy or ultrasonic energy; chemical energy (e.g. as delivered by a pharmaceutical drug or other agent); and combinations of these. Energy delivery module 360 can comprise an energy delivery module selected from the group consisting of: RF generator; light energy delivery unit; cryogenic energy delivery unit; ultrasound energy delivery unit; microwave energy delivery unit; electroporation energy delivery unit; and combinations of these. In some embodiments energy delivery module 360 comprises an RF generator configured to provide RF ablation energy to one or more energy delivery elements 130 (i.e. when each energy delivery element 130 comprises an electrode). Doses DOE1 and DOE2 can comprise similar or dissimilar forms of energy (e.g. RF energy and another form of energy).

In some embodiments, EDC 300 comprises one or more functional elements, such as functional element 309 shown and described herein.

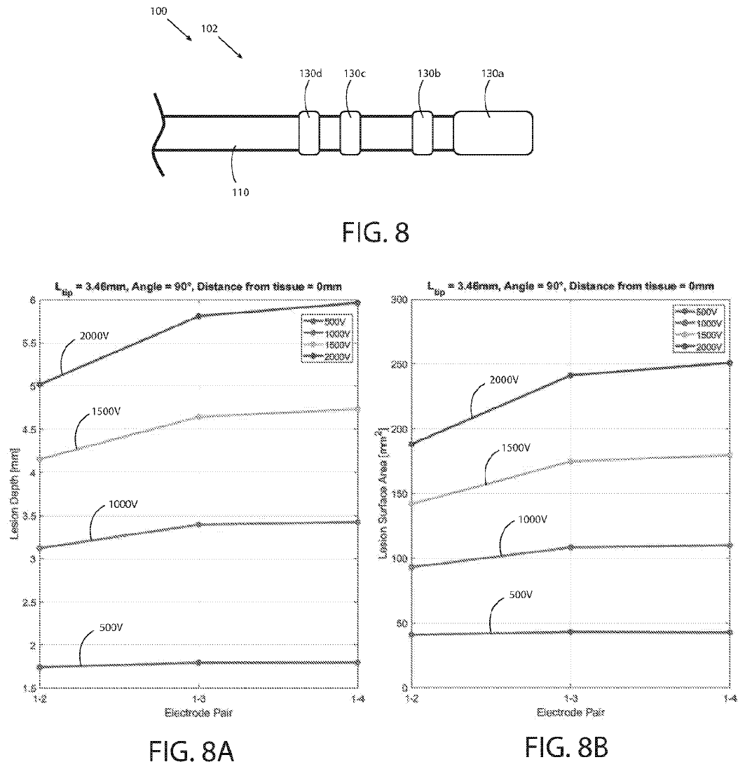

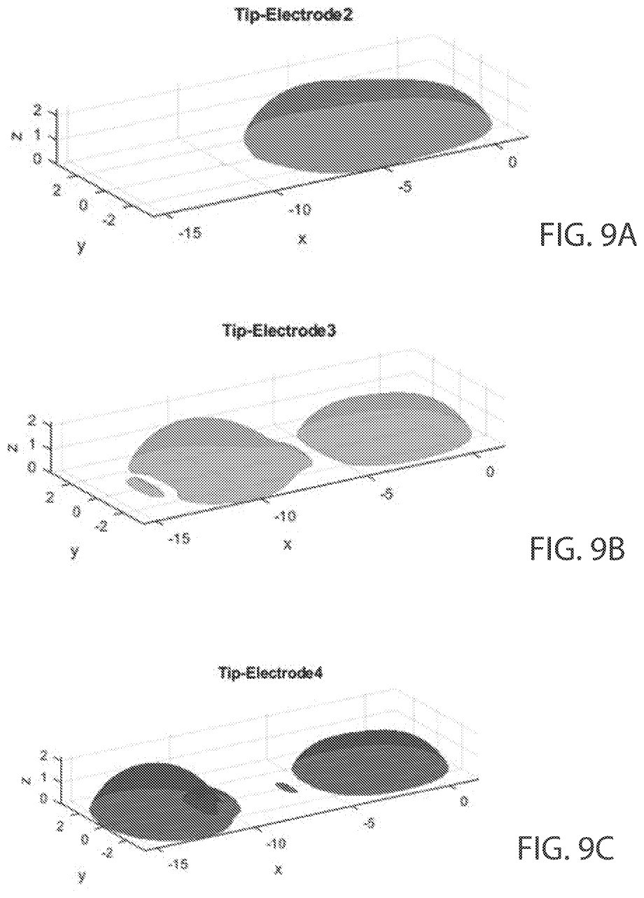

EDD 100 can comprise one or more device configured to deliver energy, such as an energy-delivering device comprising a catheter, surgical tool, laparoscopic tool, and/or endoscopic tool. EDD 100 can include shaft 110, typically a flexible shaft, including proximal end 111. EDD 100 includes distal portion 102 shown. An operator graspable portion, handle 120, can be positioned on proximal end 111 of shaft 110. Handle 120 can comprise one or more controls (e.g. one or more buttons, switches, levers, and the like), such as control 121 shown. In some embodiments, EDD 100 distal portion 102 is of similar construction and arrangement as EDD 100 of FIG. 8 described herein.

EDD 100 comprises one or more elements configured to deliver energy to tissue, such as energy delivery elements 130a-d shown in FIG. 1. In some embodiments, one or more energy delivery elements 130 is configured to deliver a first dose of energy, dose DOE1 (e.g. as provided by EDC 300 and as described herein, such as RF energy delivered in a monopolar or bipolar arrangement), and a pair of energy delivery elements 130 is configured to deliver a second dose of energy, dose DOE2 (e.g. also as provided by EDC 300 and as described herein). In some embodiments, dose DOE1 and dose DOE2 are delivered by the same set of components (e.g. the same pair of elements 130). Alternatively, energy delivery element 130 used to deliver dose DOE1 is not included in the set of elements 130 used to deliver dose DOE 2, or vice versa. In some embodiments, dose DOE 1 is delivered by one or more energy delivery elements 130 (e.g. at least element 130a), and dose DOE2 is delivered by at least two energy delivery elements 130 (e.g. at least element 130a and one or more of elements 130b-d).

Each energy delivery element 130 can comprise one or more elements configured to deliver one, two or more forms of energy selected from the group consisting of: thermal energy, such as heat energy or cryogenic energy; electromagnetic energy, such as radiofrequency (RF) energy and/or microwave energy; light energy, such as light energy provided by a laser; sound energy, such as subsonic energy or ultrasonic energy; chemical energy; and combinations of these. In some embodiments, energy delivery element 130 delivers at least two forms of energy selected from the group consisting of: thermal energy, such as heat energy or cryogenic energy; electromagnetic energy, such as radiofrequency (RF) energy and/or microwave energy; light energy, such as light energy provided by a laser; sound energy, such as subsonic energy or ultrasonic energy; chemical energy; and combinations of these. Energy delivery element 130 can comprise one or more energy delivery elements positioned on the distal portion of EDD 100, such as device distal portion 102 shown. Energy delivery element 130 can include at least one energy delivery element (e.g. at least one electrode, at least one optical element configured to deliver light energy, and/or at least one cryogenic fluid delivery element) positioned on the distal end of EDD 100, in a “tip electrode” configuration. In some embodiments, EDD 100 can comprise two, three, or more energy delivery elements 130, such as multiple electrodes configured to deliver monopolar and/or bipolar electromagnetic (e.g. RF) energy to heat, ablate, and/or otherwise affect tissue (e.g. create a desired lesion in tissue). One or more energy delivery elements 130 can each comprise an electrode, such as an electrode configured to deliver radiofrequency (RF) and/or other electromagnetic energy. Two or more energy delivery elements 130 can be configured as a pair of electrodes that delivers an irreversible electroporation pulse of energy (e.g. as provided as dose DOE2 by EDC 300). Energy delivery elements 130 can comprise one or more electrodes positioned at the end of EDD 100 (e.g. element 130a shown in FIG. 1). Energy delivery elements 130 can comprise an array of energy delivery elements (e.g. an array of electrodes), such as is shown in FIGS. 1 and 8. In some embodiments, energy delivery elements 130 comprises a return electrode pad, electrode 130′ shown in FIG. 1. Element 130′ can comprise an electrode configured as a return electrode for delivery of energy between one or more elements 130 of EDD 100 (e.g. for delivery of monopolar RF energy by EDD 100).

In some embodiments, EDD 100 comprises two or more devices for delivering energy to tissue, such as a first EDD 100′ comprising one or more energy delivery elements 130, and a second EDD 100″ comprising one or more energy delivery elements 130 (EDD 100′ and 100″ not shown, but similar or dissimilar energy delivery devices each comprising one or more delivery elements 130). In these embodiments, dose DOE1 and/or dose DOE2 can comprise a dose that is delivered between an element 130 of EDD 100′ and an element 130 of EDD 100′. For example, an RF energy dose and/or an IEP dose can be delivered between an element 130 of EDD 100′ that is positioned at a location on an endocardial surface of the heart, and an element 130 of EDD 100″ that is positioned at a location on an epicardial surface of the heart (e.g. an epicardial surface location that is relatively close to the endocardial surface location of element 130 of EDD 100′).

EDD 100 can comprise an assembly configured to measure, monitor, react to, and/or maintain a force (e.g. a force between tissue and one or more portions of EDD 100), such as force maintenance assembly 150 shown. Force maintenance assembly 150 can be positioned within handle 120, within a portion of shaft 110 (e.g. within the distal portion 102 of EDD 100), and/or on the distal end of shaft 110 (e.g. within distal portion 102 of EDD 100 as shown). Force maintenance assembly 150 can comprise one or more elements configured to provide or maintain a force, force maintenance elements 160 shown and as described herein. As examples, such force maintenance elements 160 can be or can include one or more of: a hydraulic element, a spring, a magnet, a compressible fluid, a memory material, and the like. The force maintenance elements 160 can be located at a distal end, proximal end, or intermediate portion of EDD 100, or a combination of two or more thereof. Force maintenance assembly 150 can also comprise one or more sensing elements, sensing elements 158 shown, which can take the form of and/or can include one or more sensors. In some embodiments, force maintenance assembly 150 comprises a similar construction and arrangement, and similar components, as described in applicant's co-pending U.S. patent application Ser. No. 16/335,893, titled “Ablation System with Force Control”, filed Mar. 22, 2019.

Force maintenance assembly 150 can be axially aligned with shaft 110 (e.g. a major axis of force maintenance assembly 150 is aligned with a central axis of distal portion 102), such as when assembly 150 is aligned with distal portion 102. Force maintenance assembly 150 can be configured to absorb mechanical shocks, and/or it can be configured to dynamically (e.g. dynamically and automatically) respond to movement of the heart wall or other cardiac tissue (e.g. avoiding reliance on the clinician to manually react to the movement of the endocardial surface in a cardiac ablation procedure). The force maintenance assembly 150 can allow and/or compensate for high and/or low frequency movements, various movement ranges, and the like. Force maintenance assembly 150 can be configured to compress over a “travel distance” (also referred to as the “compression distance” and equal to the distance force maintenance assembly 150 compresses when a force is applied) up to a pre-determined maximum distance (the “max compression distance” or “max travel distance”), such as a maximum distance comprising a length between 0.1 mm to 10 mm, a maximum distance comprising a length between 0.1 mm and 5 mm, and/or some other predetermined distance range and/or limit.

Force maintenance assembly 150 can be configured to provide a pre-determined force range over all or a portion of the travel distance, for example a pre-determined constant and/or variable force (e.g. a force between 0.1 gmf and 100 gmf, between 5 gmf and 30 gmf, and/or between 10 gmf and 30 gmf). In some embodiments, force maintenance assembly 150 is configured to provide a relatively constant force over all or a portion of the travel distance, for example a pre-determined constant force between 0.1 gmf and 100 gmf, such as between 5 gmf and 30 gmf, or between 10 gmf and 30 gmf. Additionally or alternatively, in some embodiments force maintenance assembly 150 is configured to provide a variable force over all or a portion of the travel distance, such as a variable force that varies within a pre-determined range of forces (e.g. a range of forces proportional to the amount compressed). For example, force maintenance assembly 150 can be configured to apply a force that varies between 5 gmf and 30 gmf, such as a force that varies between 10 gmf and 30 gmf.

As described hereabove, force maintenance assembly 150 can include one or more sensing elements or sensors, such as sensing element 158 shown, which can be configured to produce a signal correlating to the amount of compression of force maintenance assembly 150. Additionally or alternatively, sensing element 158 can be configured to produce a signal correlating to maximum compression of force maintenance assembly 150 (e.g. a maximum force achieved during compression).

Energy delivery element 130 can be positioned on the distal end of shaft 110, such as when force maintenance assembly 150 is positioned within shaft 110. Alternatively, energy delivery element 130 can be positioned on the distal end of the force maintenance assembly 150.

EDD 100 can be configured for ablation of an atria of the heart (e.g. to create one or more lesions to treat atrial fibrillation or right atrial flutter) and/or the ventricles of the heart (e.g. to treat ventricular tachycardia). For ablation of an atria, force maintenance assembly 150 can be configured with a first max compression distance, such as a distance less than or equal to 10 mm, less than or equal to 5 mm, or less than or equal to 3 mm. Alternatively, for ablation of a venticle, force maintenance assembly 150 can be configured with a second max compression distance, such as a distance greater than the first max compression distance, such as a distance at least 1 mm greater than the first max compression distance, such as a second (ventricular) max compression distance of at least 3 mm or at least 6 mm. In some embodiments, the first (atrial) max compression distance comprises a distance of approximately 2-3 mm. In some embodiments, the second (ventricular) max compression distance comprises a distance of approximately 4-6 mm.

System 10 can include at least a second energy delivery device, EDD 100′, such as a second EDD 100′ that is configured for use in the atria and/or ventricles of the heart (e.g. EDD 100′ comprises a catheter for insertion the patient's vascular system and into a chamber of the patient's heart). In some embodiments, first EDD 100 is configured for use in an atrium (e.g. and not a ventricle) and second EDD 100′ is configured for use in a ventricle (e.g. and not an atria). In these embodiments, first EDD 100 can include a force maintenance assembly 150 comprising a shorter max compression distance as compared to the max compression distance of the force maintenance assembly 150 positioned within second EDD 100′. In some embodiments, dose DOE2 comprises an IEP pulse configured to create an effective lesion in a ventricle of the heart of the patient, such as when the IEP dose is based on a voltage of no more than 5 kV. In some embodiments, dose DOE2 comprises an IEP pulse configured to create an effective lesion in an atrium of the heart of the patient, such as when the IEP dose is based on a voltage of no more than 2 kV.

EDD 100 can comprise one or more electrodes, mapping electrodes 135 shown, which can be configured to record biopotential information (e.g. cardiac electrical activity data and the like) and/or position information (e.g. data regarding EDD 100 position within the patient's anatomy). Mapping electrode 135 can comprise one or more electrodes positioned on distal portion 102 of EDD 100, as shown. Mapping electrode 135 can comprise a ring electrode. In some embodiments, mapping electrode 135 comprises at least one sensor or sensing element (“sensor” herein), such as an electrode-based sensor and/or a non-electrode based sensor (e.g. a light sensor, a temperature sensor, a pH sensor, a physiologic sensor such as a blood sensor, a blood gas sensor, and the like). In some embodiments, one or more mapping electrodes 135 and one or more energy delivery elements 130 comprise the same component.

EDD 100 is configured to operably attach to EDC 300. EDD 100 comprises one or more wires, filaments, and/or other conduits, conduit 125, and one or more attached connectors, connector 126. Connector 126 operably attaches to a mating connector, connector 301b of EDC 300. Conduit 125 can comprise one or more wires or conductive traces (“wires” herein), optical fibers, tubes (e.g. hydraulic, pneumatic, irrigation or other fluid delivery tubes), wave guides, and/or mechanical linkages (e.g. translating filament), each of which can be used to operably attach one or more components of EDC 300 to one or more components of EDD 100.

System 10 can comprise one or more functional elements, such as functional elements 119, 129, 219, 229, and/or 309 shown in FIG. 1 and described in detail herein. Functional elements 119, 129, 219, 229, and/or 309 can each comprise one or more sensors and/or one or more transducers, as described herein. In some embodiments, functional elements 119, 129, 219, 229, and/or 309 comprise a transducer selected from the group consisting of: heating element; cooling element; vibrational transducer; ultrasound transducer; electrode; light delivery element; drug or other agent delivery element; and combinations of one or more of these. In some embodiments, functional elements 119, 129, 219, 229, and/or 309 comprise a sensor selected from the group consisting of: a physiologic sensor; a blood pressure sensor; a blood gas sensor; a pressure sensor; a strain gauge; a force sensor; a chemical sensor; an impedance sensor; a magnetic sensor; an electrode; a displacement sensor (e.g. a sensor configured to determine the distance force maintenance assembly 150 is compressed); a flow sensor; and combinations of one or more of these. In some embodiments, functional elements 129 and/or 229 comprise functional elements configured to provide feedback, and/or otherwise alert the user to the status of one or more components of system 10 (e.g. when an undesired condition is present). Functional elements 129 and/or 229 can comprise an element selected from the group consisting of: a haptic transducer; a light source, such as an LED light source; an audio transducer, such as a speaker; and combinations of one or more of these.

Mapping catheter 200 of system 10 includes shaft 210, typically a flexible shaft comprising one or more lumens. Positioned on distal end 213 as shown, or positioned at least on a distal portion of shaft 210, is basket assembly 230. An operator graspable portion, handle 220, is positioned on proximal end 211 of shaft 210. Handle 220 can comprise one or more controls, such as control 221 shown.

Basket assembly 230 can comprise an expandable assembly, such as an assembly resiliently biased in a radially expanded or compacted state and configured to correspondingly be compacted or expanded, respectively, such as via control 221, by advancing out of the distal end of a sheath (to radially expand), and/or by being retracted within a sheath (to radially compact), such as sheath 12 or the like. Basket assembly 230 comprises an array of filaments, splines 231, which can comprise metal (e.g. stainless steel and/or nickel titanium alloy) and/or plastic filaments that are resiliently biased (e.g. biased in an expanded and/or compacted state). Basket assembly 230 can include a plurality of electrodes, electrodes 232, which are coupled to splines 231. Additionally or alternatively, basket assembly 230 can include a plurality of ultrasound transducers, transducers 233 which can also be coupled to splines 231. In some embodiments, basket assembly 230 and/or mapping catheter 200 are of similar construction and arrangement to the similar components described in applicant's co-pending U.S. patent application Ser. No. 16/389,006, titled “Device and Method For the Geometric Determination of Electrical Dipole Densities on the Cardiac Wall”, filed Apr. 19, 2019 and/or applicant's co-pending U.S. patent application Ser. No. 16/242,810, titled “Expandable Catheter Assembly with Flexible Printed Circuit Board (PCB) Electrical Pathways”, filed Jan. 8, 2019. In some embodiments, one or more electrodes 232 and/or ultrasound transducers 233 additionally or alternatively include a sensor, such as a physiologic sensor and/or other sensor as described herein.

Mapping catheter 200 of system 10 can comprise one or more functional elements, such as functional elements 219, 229 shown, and described herein. In some embodiments, one or more functional elements 219 and/or 229 are positioned on basket assembly 230 (e.g. on one or more splines 231).

Mapping catheter 200 can be configured to operably attach to EDC 300. Mapping catheter 200 comprises one or more wires, filaments, and/or other conduits, conduit 225, and one or more attached connectors, connector 226, each as shown. Connector 226 operably attaches to a mating connector, connector 301a of EDC 300. Conduit 225 can comprise one or more wires, optical fibers, tubes (e.g. hydraulic, pneumatic, irrigation or other fluid delivery tubes), wave guides, and/or mechanical linkages (e.g. translating filament), each of which can be used to operably attach one or more components of EDC 300 to one or more components of mapping catheter 200.

System 10 can include one or more patch electrodes, patch electrode 60 shown, which can comprise standard skin electrodes and/or other electrodes configured to attach to the skin of the patient and transmit electrical signals through the patient and/or receive electrical signals from the patient. In some embodiments, patch electrodes 60 are configured to record the patient's electrocardiogram (ECG) and/or to transmit and/or receive localization signals of system 10. Patch electrodes 60 can be configured to operably attach (e.g. electrically attach) to EDC 300. Each patch electrode 60 can comprise one or more conduits, conduit 65, (e.g. one or more electrical wires), and one or more attached connectors, connector 66. Connector 66 operably attaches to a mating connector, connector 301c of EDC 300.

EDC 300 includes one or more internal components configured to control and/or otherwise interface with the one or more energy delivery devices, EDD 100, one or more mapping catheters 200, and/or one or more patient patches 60. EDC 300 comprises one or more wires, filaments, and/or other conduits, conduits 302, such as conduits 302a, 302b, and/or 302c, which via connectors 301a, 301b, and/or 301c operably connect to the one or more EDDs 100, one or more mapping catheters 200, and/or one or more patient patches 60, respectively. Conduits 302 can comprise one or more wires, optical fibers, tubes (e.g. hydraulic, pneumatic, irrigation or other fluid delivery tubes), wave guides, and/or mechanical linkages (e.g. translating filament).

EDC 300 can comprise a patient interface unit, PIU 310. PIU 310 can be connected (e.g. electrically connected) to one or more of units 320, 330, 340, 350, 360, and/or 370, each described in detail herein, via bus 305. Bus 305 can comprise one or more wires, optical fibers, and/or other conduits configured to provide power, transmit data, and/or receive data. In some embodiments, bus 305 comprises one or more fluid delivery tubes configured to provide hydraulic fluid, irrigation fluid, and/or other fluid as described herein. PIU 310 can be operably attached to units 340, 350, 360, 330 and/or 320, such as to allow power, data, fluids, and/or mechanical linkages to pass between PIU 310 and one or more of: EDD 100, mapping catheter 200, and/or patches 60. In some embodiments, PIU 310 can reduce undesired electrical interaction between two or more modules of EDC 300. For example, PIU 310 can include one or more filters (e.g. one, two or more parallel LC notch filters and/or low-pass filters) configured to reduce electrical interference between a mapping module and an RF generator, such as interference from signals transmitted into and received from the patient. PIU 310 can include one or more components selected from the group consisting of: a filter; a transformer; a buffer; an amplifier; a pass thru (e.g. a conduit that is unfiltered or otherwise unaltered by PIU 310, such as a fluid conduit); and combinations of one or more of these. In some embodiments, PIU 310 comprises an electrical protection circuit configured to protect EDC 300 from damage caused by high-energy signals such as defibrillation pulses and/or RF ablation energy delivered to the patient.

EDC 300 can comprise a clinician or other user interface, user interface unit 320 shown, which includes one or more user input and/or user output components. In some embodiments, user interface unit 320 comprises a joystick, keyboard, mouse, touchscreen, and/or other human interface device, such as HID 321 shown. In some embodiments, user interface unit 320 comprises a display, such as display 322, also shown.

EDC 300 can comprise a signal processing assembly, processor 330. In some embodiments, processor 330 comprises one or more algorithms, such as algorithm 335 shown. Processor 330 can receive a signal, such as a signal from one or more sensors (as described herein) of EDD 100 and/or mapping catheter 200. Processor 330 can be configured to perform one or more mathematical operations on the received signal, and produce a result correlating to a quantitative or qualitative measure of the force applied by EDD 100 to tissue, the amount of compression of force maintenance assembly 150, the orientation of EDD 100, the proximity of a portion of EDD 100 to cardiac tissue, and/or the level or quality of contact between a portion of EDD 100 and cardiac tissue. The one or more mathematical operations can comprise an operation of function selected from the group consisting of: arithmetic operations; statistical operations; linear and/or non-linear functions; operations as a function of time; operations as a function of space or distance; comparison to a threshold; comparison to a range; and combinations of one or more of these. In some embodiments, algorithm 335 comprises a machine learning or other artificial intelligence (AI) algorithm. In some embodiments, algorithm 335 is configured to monitor, assess and/or control (“control” herein) force maintenance assembly 150 (e.g. adjust one or more parameters of force maintenance assembly in a closed loop or semi-closed loop fashion), such as control based on the sensor signal. In some embodiments, algorithm 335 is configured to determine and/or assess at least one of contact, force, or pressure applied by EDD 100 to tissue. In some embodiments, algorithm 335 processes one or more signals received from one or more sensors of system 10, such as a signal correlating to: the temperature of the energy delivery element; the temperature of the tissue surrounding the energy delivery element; the duration of energy delivery to tissue; the level of energy being delivered to tissue; the force and/or pressure being applied to tissue; and combinations of one or more of these. Algorithm 335 can be configured to modify the energy delivery based on these signals, for example to stop the energy delivery when a combination of sufficient parameter levels has been reached, for example when a sufficient energy delivery at a sufficient pressure for a sufficient period of time has been reached. In some embodiments, system 10 is configured to deliver increased energy levels to decrease duration of energy delivery to tissue. Alternatively or additionally, system 10 can be configured to increase duration of energy delivery to tissue, in order to decrease an energy level. In some embodiments, system 10 controls force between one or more energy delivery elements 130 and tissue to adjust one or more of duration of energy delivery and/or level of energy delivery (e.g. voltage level, current level and/or power level). In some embodiments, system 10 adjusts duration of energy delivery and/or level of energy delivery based on a measured and/or controlled level of force between one or more energy delivery elements 130 and tissue.

In some embodiments, algorithm 335 (e.g. an AI algorithm) is configured to define, adjust, and/or otherwise control the delivery of energy by EDC 300 to EDD 100, such as to control dose DOE1 and/or DOE2. In some embodiments, algorithm 335 is configured to modify (e.g. in a closed loop arrangement) the delivery of dose DOE1 and/or DOE2, such as a modification based on tissue impedance and/or other physiologic parameter of the patient. In some embodiments, algorithm 335 is configured to modify dose DOE2 based on a parameter (e.g. a measured parameter) associated with a previously delivered dose DOE1.

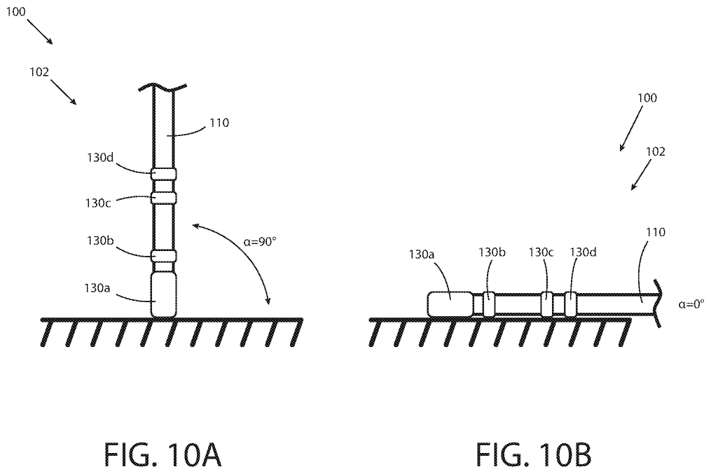

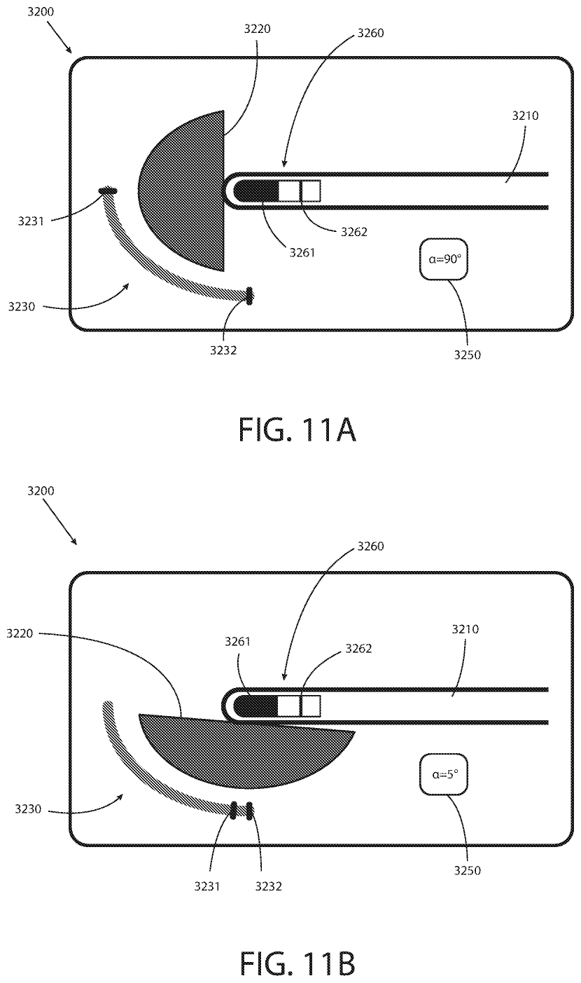

Algorithm 335 can be used to determine the orientation angle of an EDD 100, such as a determination based on data provided by a sensor of system 10, and/or by a separate imaging device, as described herein in reference to FIGS. 10-11.

Algorithm 335 can be used to determine electric field strength, such as an electric field used to perform pulsed field ablation as described herein in reference to FIGS. 10-13.

Algorithm 335 can be used to provide lesion information, such as a predicted size (e.g. length, width, depth, and/or volume) of a lesion to be created, such as is described herein in reference to FIGS. 10-13.

EDC 300 can comprise a fluid delivery module, module 370 shown, which can be configured to deliver a fluid (e.g. a hydraulic fluid and/or an irrigation fluid as described herein), to EDD 100 and/or mapping catheter 200, such as via PIU 310 as shown. In an alternative embodiment, fluid delivery module 370 is connected to EDD 100 and/or mapping catheter 200 without passing through PIU 310. Fluid delivery module 370 can comprise one or more fluid delivery devices (e.g. peristaltic pump, syringe pump, gravity-feed flow controller and/or other fluid delivery device) which can be attached to one or more sources of saline and/or other fluid, fluid 70 shown.

Fluid 70 can comprise a fluid of a known conductivity (e.g. a relatively low conductivity and/or a conductivity at least less than that of blood), such as a fluid delivered to steer current and/or steer an electromagnetic field (e.g. to surround one or more electrodes, such as during the delivery of energy to cause pulsed field ablation of target tissue).

EDC 300 can comprise a force maintenance module, module 340 shown. Force maintenance module 340 can be configured to provide a signal that allows system 10 to adjust force applied by EDD 100 to tissue, such as to provide a control signal to force maintenance assembly 150. In some embodiments, force maintenance module 340 is configured to deliver and/or at least control (e.g. control the pressure of) a supply of hydraulic fluid to EDD 100 (e.g. via fluid delivery module 370).

In some embodiments, force maintenance module 340 is configured to automatically adjust force between an electrode 130 and tissue, such as to create a desired electric field during pulsed field ablation of target tissue.

EDC 300 can comprise a mapping module, module 350 shown. In some embodiments, mapping module 350 comprises a module configured to record and/or process ultrasound information, such as ultrasound module 351 shown. In some embodiments, mapping module 350 comprises a module configured to record and process biopotential information, such as biopotential module 352 shown. Mapping module 350 can transmit energy and/or signals to EDD 100, mapping catheter 200, and/or patches 60 via PIU 310 (as shown), or otherwise. Mapping module 350 can be configured to transmit one or more signals into the patient (e.g. via one or more patches 60), such as to create a localization field within the patient. Furthermore, mapping module 350 can receive signals from one or more electrodes (or other sensors) of EDD 100 and/or mapping catheter 200, such as signals correlating to the localization signals, such as to determine the localization of the one or more electrodes within the localization field (e.g. to determine the location and/or orientation of the associated catheter(s) within the patient). In some embodiments, two or more localization fields can be used simultaneously. The components used to generate and/or sense the localization fields (e.g. patches 60 and/or the one or more electrodes of EDD 100 or mapping catheter 200), can be configured to transmit localization signals (herein “source”), receive localization signals (herein “sink”), and/or transmit and receive localization signals interchangeably. For example, the components can be multiplexed to source and sink localization signals between each other in a pattern configured to enhance the localization information received by mapping module 350, such as information regarding the relative position between a component of system 10 and the cardiac tissue or other structures within the cardiac chamber and/or another component of system 10.

For example, the direction of current flow between two or more components used to perform a localization measurement can be reversed. For example, in an impedance-based system, multiple (e.g. 3 or 4) localization fields can be generated simultaneously using multiple frequency ranges. All electrodes and/or sensors within the field can be used to sense the localization field. The components used to source (e.g. transmit the localization signals) and sink (e.g. sense the localization signals) the localization fields can be fixed and static, such as patches 60 positioned on the body surface that are used to source the localization fields, and electrodes located on one or more components of system 10 and positioned within the patient that are used to sink the localization signals. Alternatively, the components can be time-multiplexed and/or frequency-multiplexed, such as by sourcing and sinking current from different sets of components at various frequencies and/or at various times. As an example of a time-multiplexed localization method, the system can include three source/sink components, A-C. In a first configuration, component A is used to source, and component B is used to sink. In a second configuration, B can be used to source and A to sink. In a third configuration, C is used to source, and B is used to sink. These three configurations can be multiplexed to provide an enhanced localization method. Using all possible permutations would provide the full complement of information available via the source-sink configurations. Subsets of these configurations can be selected to reduce electronic and algorithmic complexity, while providing sufficient information to resolve the number of conditions and/or states required. In some embodiments, the electronics are configured to minimize current leakage (e.g. paths to a ground) within a range of frequencies (such as 10-100 kHz) via sensors and/or electrodes present within the localization field and/or used to measure the localization field. For example, current leakage can be minimized with the design of a sufficiently high input impedance in the localization frequency range of interest.

In some embodiments, ultrasound module 351 of mapping module 350 is configured to transmit and receive ultrasound signals via one or more ultrasound transducers 233 of mapping catheter 200 to determine the distance between ultrasound transducers 233 and the cardiac tissue, such as to, in coordination with the localization data, generate an anatomical model of the cardiac tissue. Biopotential module 352 of mapping module 350 can be configured to record one or more biopotential signals, such as via electrodes 232 of mapping catheter 200, to create an electrical activity map of the cardiac chamber. In some embodiments, mapping module 350, including ultrasound module 351 and biopotential module 352, are of similar construction and arrangement to the similar components described in applicants co-pending U.S. patent application Ser. No. 15/569,185, titled “Ultrasound Sequencing System and Method”, filed Oct. 25, 2017, and/or applicant's co-pending U.S. patent application Ser. No. 16/849,045, titled “Localization System and Method Useful in the Acquisition and Analysis of Cardiac Information”, filed Apr. 15, 2020.

One or more sensors of EDD 100 (e.g. one or more of functional elements 119 or 129 configured as one or more sensors, and/or other sensors as described herein) can be configured to produce a signal correlating to a level of contact between one or more energy delivery elements 130 and tissue (e.g. cardiac tissue). The signal provided can simply differentiate a minimum (sufficient) level of contact versus an insufficient level of contact (e.g. a lack of contact), and/or it can provide data that differentiates various levels of contact (e.g. a quantitative assessment of force between one or more energy delivery elements 130 and tissue). EDC 300 can provide qualitative and/or quantitative contact information to a user (e.g. a clinician), such as via display 322, the information indicative of the level of contact between one or more energy delivery elements 130 and tissue (e.g. chamber wall and/or other cardiac tissue). In some embodiments, system 10 is configured to provide (via display 322) information comprising: sufficient contact achieved (e.g. sufficient contact to perform a efficacious delivery of energy to tissue); insufficient contact achieved; level of force achieved; level of pressure achieved; distance or proximity to a boundary; orientation or angle-of-attack to a boundary or other tissue location; topology of a proximate boundary; contact efficiency; and combinations of one or more of these.

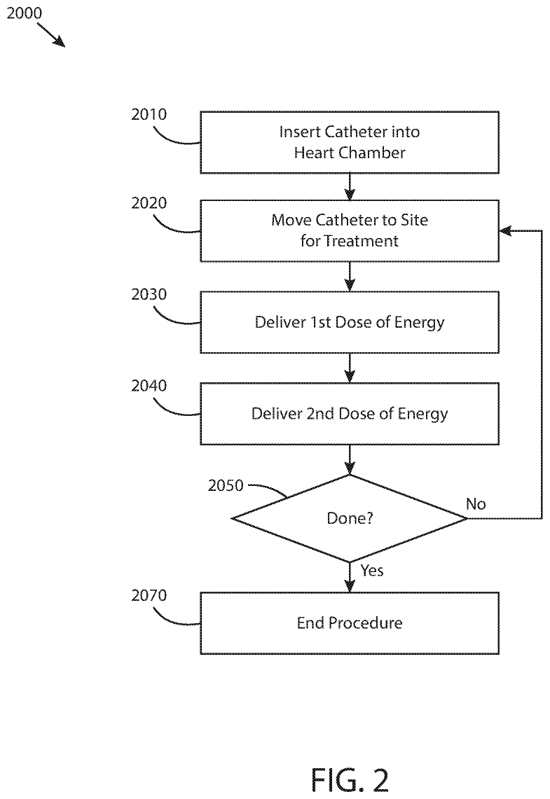

The tissue treatment methods described herebelow in reference to FIGS. 2-7 are described in reference to a catheter, such as energy delivery device EDD 100 described herein, delivering two forms of energy to target tissue of the patient. It should be considered within the spirit and scope of this application that other types of energy delivery devices could be used, such as surgical tools, laparoscopic tools, endoscopic tools, and/or other energy delivery tools. The methods described herebelow in reference to FIGS. 2-7 are described in reference to target tissue comprising heart tissue, such as heart chamber tissue in which energy is delivered to an endocardial surface of the heart. It should be considered within the spirit and scope of this application that energy can alternatively be delivered within a heart wall and/or to an epicardial surface, and that other tissues of the patient could be treated with the systems, devices, and methods of the present inventive concepts. Doses of energy dose DOE1 and dose DOE2 described in reference to FIGS. 2-7 can comprise similar forms of energy (e.g. where both comprise RF energy delivery), or different forms of energy (e.g. where dose DOE2 comprises RF or other electromagnetic energy delivery and dose DOE1 comprises non-electromagnetic energy delivery). Dose DOE1 can comprise a delivery of energy configured to reversibly warm target tissue, while dose DOE2 can comprise a delivery of energy configured to irreversibly electroporate the target tissue, such as via delivery of an IEP as described herein, such as to create a desired lesion in tissue (e.g. to treat AF or other arrhythmia of the patient). This pre-warming of tissue can provide numerous advantages, as described herein, such as when dose DOE2 comprises a delivery of energy (e.g. RF energy) with a lower amplitude than that which would have been necessary to irreversibly electroporate the target tissue if the target tissue was at body temperature (e.g. not pre-warmed by dose DOE1).

In some embodiments, EDC 300 is configured as a monitoring device, such as when one or more sensors of system 10 provide physiologic information of the patient and/or information related to the patient's environment. In some embodiments, EDC 300 configures dose DOE1 and/or dose DOE2 based on this information. For example, EDC 300 can be configured to provide dose DOE1 and/or dose DOE2 based on patient physiologic information selected from the group consisting of: cardiac cycle; heart rate; blood pressure; blood flow rate; respiration rate; brain activity; electrogram amplitude (e.g. as measured in unipolar and/or bipolar modes); tissue impedance; and combinations of these.

Referring now to FIG. 2, a flow chart of a method of delivering energy to tissue is illustrated, consistent with the present inventive concepts. Method 2000 is described using system 10 and its components, as described herein.

In STEP 2010, the distal portion of EDD 100 is inserted into a heart chamber of a patient. In some embodiments, the distal portion of catheter 200 is also inserted into a heart chamber of the patient, such as to provide biopotential, anatomical visualization, and/or other cardiac mapping functions.

In STEP 2020, one or more energy delivery elements 130 of EDD 100 are moved proximate a tissue site for treatment, “target tissue” herein.

In STEP 2030, a first dose of energy, dose DOE1 as described herein, is provided by EDC 300 to EDD 100 and delivered to the target tissue by the one or more energy delivery elements 130.

In STEP 2040, a second dose of energy, dose DOE2 as described herein, is provided by EDC 300 to EDD 100 and delivered to the target tissue by the one or more energy delivery elements 130 (e.g. the same and/or different energy delivery elements 130 that delivered dose DOE1 in STEP 2030).

In STEP 2050, a check of procedure completeness is performed. If the procedure is not complete, such as when additional tissue (e.g. additional target tissue) is to be treated, the method returns to STEP 2020. If the procedure is complete, the procedure is ended in STEP 2070.

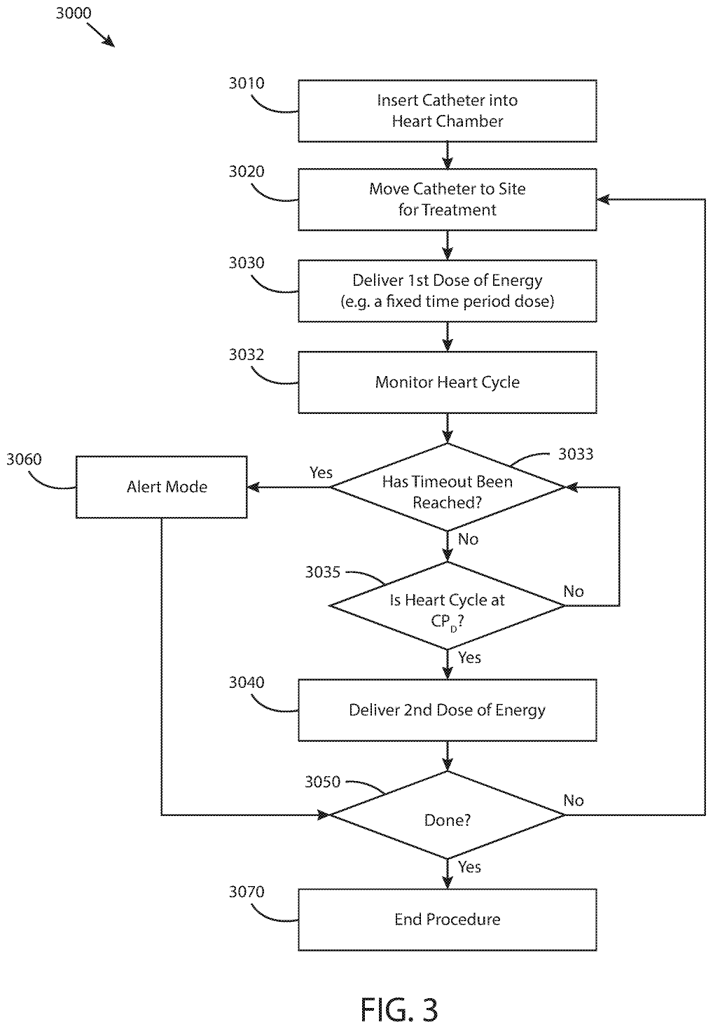

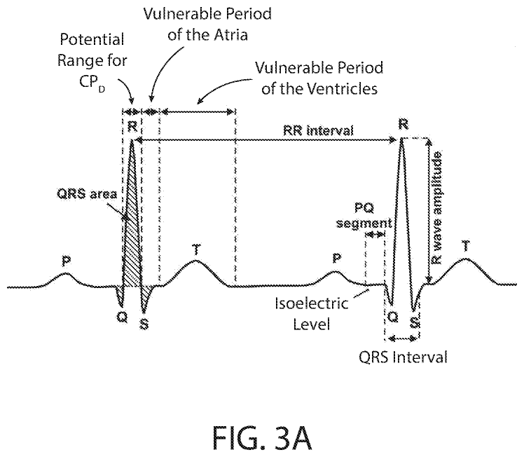

Referring now to FIG. 3, a flow chart of a method of delivering energy to tissue is illustrated, consistent with the present inventive concepts. Method 3000 is described using system 10 and its components, as described herein.

In STEP 3010, the distal portion of EDD 100 is inserted into a heart chamber of a patient. In some embodiments, the distal portion of catheter 200 is also inserted into a heart chamber of the patient, such as to provide biopotential, anatomical visualization, and/or other cardiac mapping functions.

In STEP 3020, one or more energy delivery elements 130 of EDD 100 are moved proximate target tissue.

In STEP 3030, a first dose of energy, dose DOE1 as described herein, is provided by EDC 300 to EDD 100 and delivered to target tissue by the one or more energy delivery elements 130. In some embodiments, the dose DOE1 comprises delivery of energy (e.g. RF energy) for a fixed time period.

In STEP 3032, the heart cycle of the patient is monitored, such as by EDC 300 or other component of system 10.

In STEP 3033, a check of reaching a “timeout” is performed, such as a timeout period comprising the time since the dose DOE1 delivery finished. If the timeout period has been reached, STEP 3060 is performed in which system 10 enters an alert mode, and method 3000 continues to STEP 3050 described herebelow. If the timeout period has not been reached, STEP 3035 is performed.

In STEP 3035, a check to determine if the patient's heart cycle is at a desired cycle point, cycle point CPD. If the patient's heart cycle is not at point CPD, method 3000 returns to STEP 3033. If the patient's heart cycle is at point CPD, STEP 3040 is performed.

In STEP 3040, a second dose of energy, dose DOE2 as described herein, is provided by EDC 300 to EDD 100 and delivered to target tissue by the one or more energy delivery elements 130 (e.g. the same and/or different energy delivery elements 130 that delivered dose DOE1 in STEP 3030).

In some embodiments, cycle point CPD is selected in STEP 3035 such that in STEP 3040 dose DOE2 is delivered at a time between 50 msec and 200 msec after the R wave occurs, reference FIG. 3A.

In STEP 3050, a check of procedure completeness is performed. If the procedure is not complete, such as when additional tissue (e.g. additional target tissue) is to be treated, the method returns to STEP 3020. If the procedure is complete, the procedure is ended in STEP 3070.

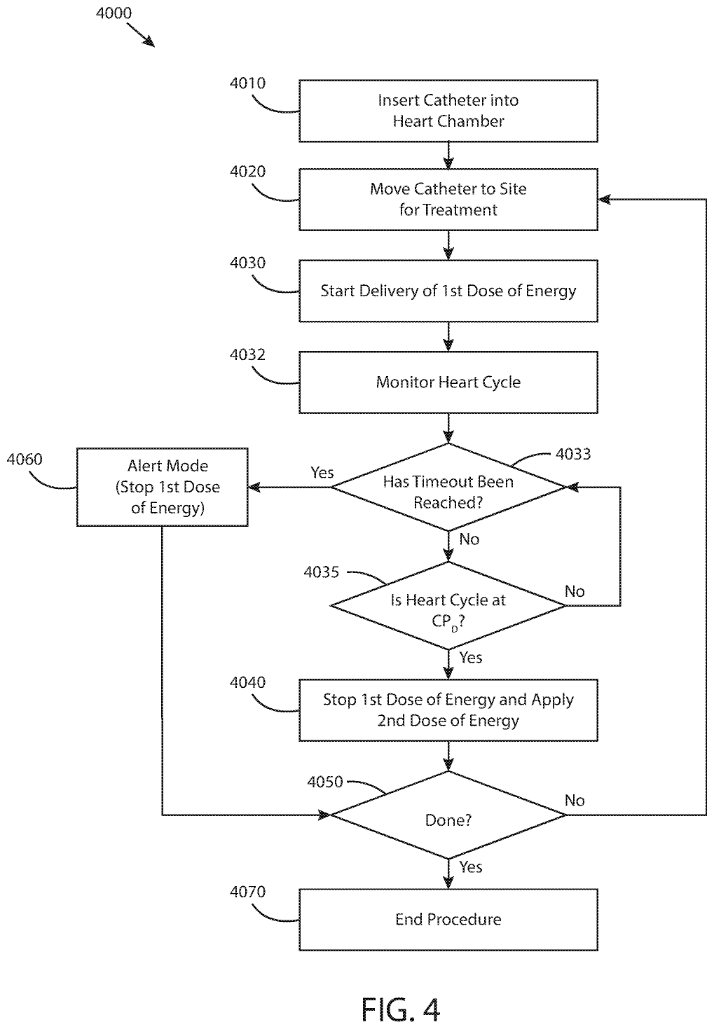

Referring now to FIG. 4, a flow chart of a method of delivering energy to tissue is illustrated, consistent with the present inventive concepts. Method 4000 is described using system 10 and its components, as described herein.

In STEP 4010, the distal portion of EDD 100 is inserted into a heart chamber of a patient. In some embodiments, the distal portion of catheter 200 is also inserted into a heart chamber of the patient, such as to provide biopotential, anatomical visualization, and/or other cardiac mapping functions.

In STEP 4020, one or more energy delivery elements 130 of EDD 100 are moved proximate target tissue.

In STEP 4030, delivery of a first dose of energy, dose DOE1 as described herein, is initiated. Energy is provided by EDC 300 to EDD 100 and delivered to target tissue by the one or more energy delivery elements 130.

In STEP 4032, the heart cycle of the patient is monitored, such as by EDC 300 or other component of system 10.

In STEP 4033, a check of reaching a “timeout” is performed, such as a timeout period comprising the time since the dose DOE1 delivery finished. If the timeout period has been reached, STEP 4060 is performed in which system 10 enters an alert mode, delivery of dose DOE1 is stopped, and method 4000 continues to STEP 4050 described herebelow. If the timeout period has not been reached, STEP 4035 is performed.

In STEP 4035, a check to determine if the patient's heart cycle is at a desired cycle point, cycle point CPD. If the patient's heart cycle is not at point CPD, method 4000 returns to STEP 4033. If the patient's heart cycle is at point CPD, STEP 4040 is performed.

In STEP 4040, delivery of dose DOE1 is stopped, and a second dose of energy, dose DOE2 as described herein, is provided by EDC 300 to EDD 100 and delivered to target tissue by the one or more energy delivery elements 130 (e.g. the same and/or different energy delivery elements 130 that delivered dose DOE1 in STEP 4030).

In STEP 4050, a check of procedure completeness is performed. If the procedure is not complete, such as when additional tissue (e.g. additional target tissue) is to be treated, the method returns to STEP 4020. If the procedure is complete, the procedure is ended in STEP 4070.

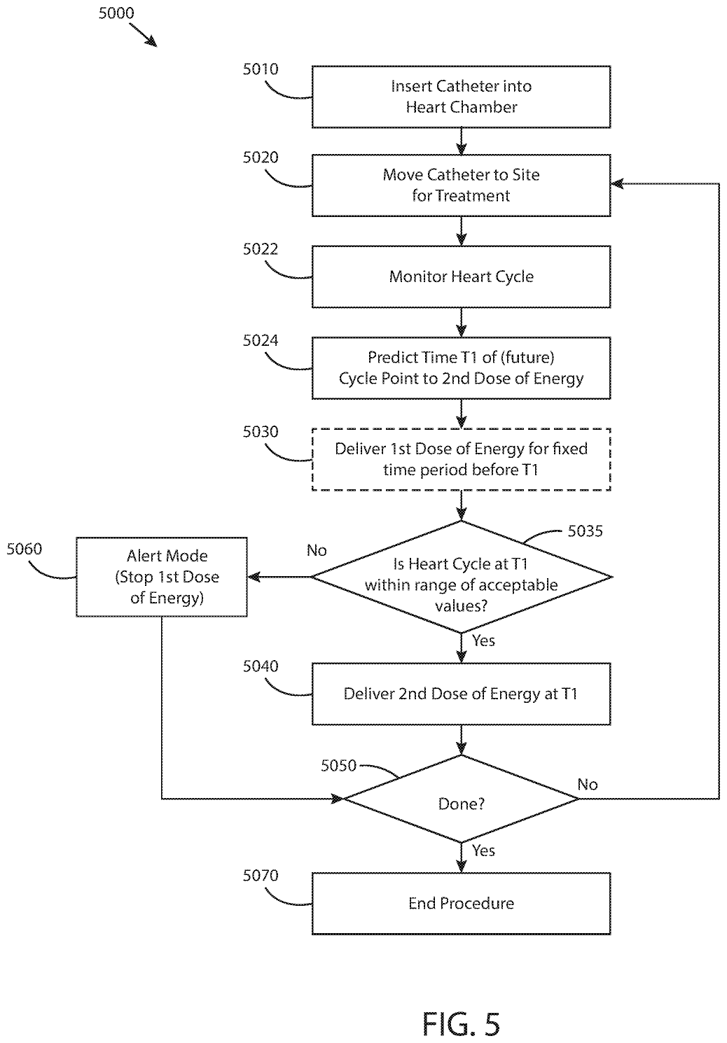

Referring now to FIG. 5, a flow chart of a method of delivering energy to tissue is illustrated, consistent with the present inventive concepts. Method 5000 is described using system 10 and its components, as described herein.

In STEP 5010, the distal portion of EDD 100 is inserted into a heart chamber of a patient. In some embodiments, the distal portion of catheter 200 is also inserted into a heart chamber of the patient, such as to provide biopotential, anatomical visualization, and/or other cardiac mapping functions.

In STEP 5020, one or more energy delivery elements 130 of EDD 100 are moved proximate target tissue.

In STEP 5022, the heart cycle of the patient is monitored, such as by EDC 300 or other component of system 10.

In STEP 5024, a time T1 of a future desired heart cycle point CPD is predicted by system 10 (e.g. via algorithm 335).

In STEP 5030, an optional step of delivering a first dose of energy, dose DOE1 as described herein, can be provided by EDC 300 to EDD 100 and delivered to target tissue by the one or more energy delivery elements 130. In some embodiments, the dose DOE1 comprises delivery of energy (e.g. RF energy) for a fixed time period (e.g. a fixed time period that ends prior to time T1).

In STEP 5035, a check is performed (e.g. at time T1 or just prior) to determine if the patient's heart cycle at time T1 is at a desired cycle point, cycle point CPD. If the patient's heart cycle is at point CPD, STEP 5040 is performed. If the patient's heart cycle is not at point CPD, STEP 5060 is performed in which system 10 enters an alert mode, delivery of dose DOE1 is stopped (if being delivered via optional step 5030), and method 5000 continues to STEP 5050 described herebelow.

In STEP 5040, a dose of energy (e.g. a first or second dose of energy), dose DOE2 as described herein, is provided by EDC 300 to EDD 100 and delivered to target tissue by the one or more energy delivery elements 130 (e.g. the same and/or different energy delivery elements 130 that may have delivered dose DOE1 in optional STEP 5030).

In STEP 5050, a check of procedure completeness is performed. If the procedure is not complete, such as when additional tissue (e.g. additional target tissue) is to be treated, the method returns to STEP 5020. If the procedure is complete, the procedure is ended in STEP 5070.

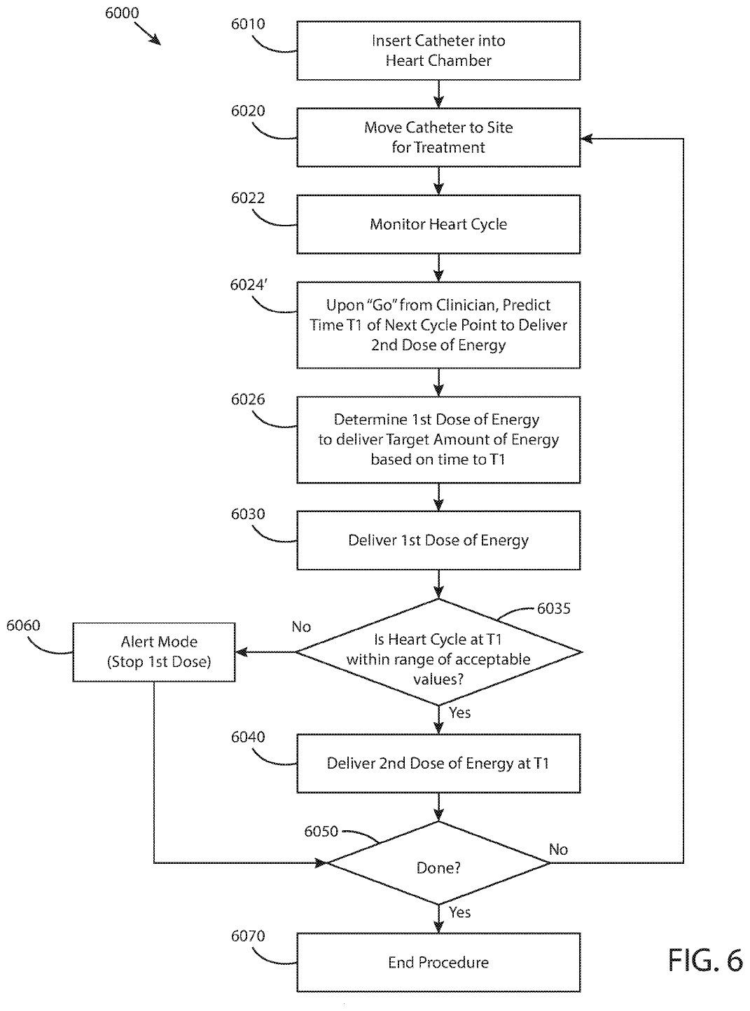

Referring now to FIG. 6, a flow chart of a method of delivering energy to tissue is illustrated, consistent with the present inventive concepts. Method 6000 is described using system 10 and its components, as described herein.

In STEP 6010, the distal portion of EDD 100 is inserted into a heart chamber of a patient. In some embodiments, the distal portion of catheter 200 is also inserted into a heart chamber of the patient, such as to provide biopotential, anatomical visualization, and/or other cardiac mapping functions.

In STEP 6020, one or more energy delivery elements 130 of EDD 100 are moved proximate target tissue.

In STEP 6022, the heart cycle of the patient is monitored, such as by EDC 300 or other component of system 10.

In STEP 6024′, after a “go” signal (e.g. an initiation request) is received from an operator of system 10 (e.g. the patient's clinician as provided via user interface 320 of EDC 300), a time T1 of a future desired heart cycle point CPD is predicted by system 10 (e.g. via algorithm 335).

In STEP 6026, a first dose of energy, dose DOE1 is determined to achieve a target amount of energy (e.g. a target amount of Joules) that is to be delivered by time T1 (e.g. delivered continuously to time T1 and/or in intermittent pulses up till time T1). This target amount of energy can be determined by system 10 and/or a clinician of the patient.

In STEP 6030, dose DOE1 as described herein and defined in STEP 6026, is provided by EDC 300 to EDD 100 and delivered to target tissue by the one or more energy delivery elements 130.

In STEP 6035, a check is performed (e.g. at time T1 or just prior) to determine if the patient's heart cycle at time T1 is at a desired cycle point, cycle point CPD. If the patient's heart cycle is at point CPD, STEP 6040 is performed. If the patient's heart cycle is not at point CPD, STEP 6060 is performed in which system 10 enters an alert mode, delivery of dose DOE1 is stopped, and method 6000 continues to STEP 6050 described herebelow.

In STEP 6040, a second dose of energy, dose DOE2 as described herein, is provided by EDC 300 to EDD 100 and delivered to target tissue by the one or more energy delivery elements 130 (e.g. the same and/or different energy delivery elements 130 that delivered dose DOE1 in STEP 6030).

In STEP 6050, a check of procedure completeness is performed. If the procedure is not complete, such as when additional tissue (e.g. additional target tissue) is to be treated, the method returns to STEP 6020. If the procedure is complete, the procedure is ended in STEP 6070.

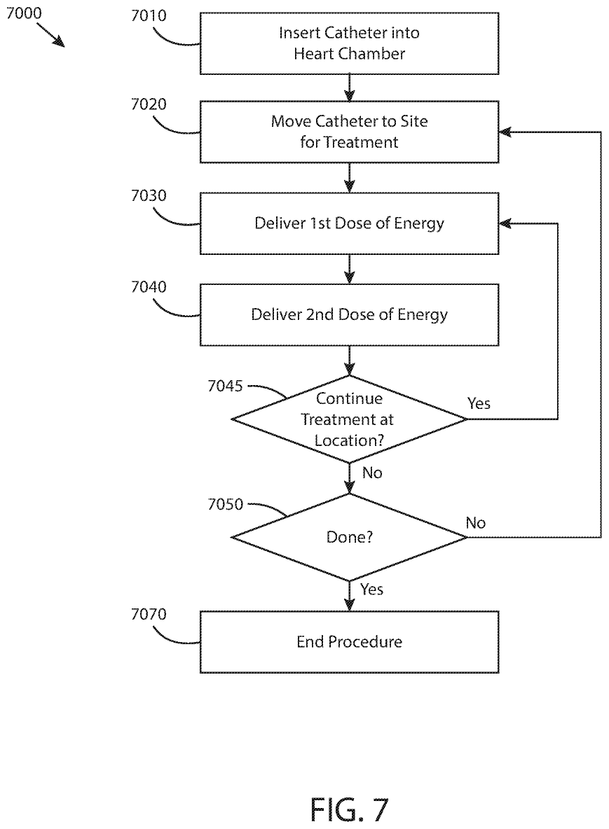

Referring now to FIG. 7, a flow chart of a method of delivering energy to tissue is illustrated, consistent with the present inventive concepts. Method 7000 is described using system 10 and its components, as described herein.

In STEP 7010, the distal portion of EDD 100 is inserted into a heart chamber of a patient. In some embodiments, the distal portion of catheter 200 is also inserted into a heart chamber of the patient, such as to provide biopotential, anatomical visualization, and/or other cardiac mapping functions.

In STEP 7020, one or more energy delivery elements 130 of EDD 100 are moved proximate target tissue.

In STEP 7030, dose DOE1 as described herein, is provided by EDC 300 to EDD 100 and delivered to target tissue by the one or more energy delivery elements 130.

In STEP 7040, a second dose of energy, dose DOE2 as described herein, is provided by EDC 300 to EDD 100 and delivered to target tissue by the one or more energy delivery elements 130 (e.g. the same and/or different energy delivery elements 130 that delivered dose DOE1 in STEP 7030).

In STEP 7045, a check of whether additional energy should be delivered to the current target tissue is performed. If additional energy delivery is to be delivered (e.g. as determined manually by an operator of system 10 and/or automatically by algorithm 335, such as when algorithm 335 comprises an AI algorithm), Method 7000 returns to repeat STEP 7030. Otherwise, STEP 7050 is performed.

In STEP 7050, a check of procedure completeness is performed. If the procedure is not complete, such as when additional tissue (e.g. additional target tissue) is to be treated, the method returns to STEP 7020. If the procedure is complete, the procedure is ended in STEP 7070.

In Method 7000 of FIG. 7, the delivery of energy in STEPS 7030 and 7040 can comprise delivering of doses DOE1 and DOE2 in an interleaving arrangement (e.g. at least a portion of dose DOE1 is delivered during STEP 7040 and/or at least a portion of dose DOE2 is delivered during STEP 7030). For example, a portion of dose DOE1 can be delivered during a portion of dose DOE2 or vice versa. In some embodiments, dose DOE1 comprises energy (e.g. RF energy) delivered in pulsed arrangement, such as when dose DOE1 comprises one or more periods of “on time” (DOE1TP_{ON}), separated by “off time” periods (DOE1TP_{OFF}), where energy is delivered during DOE1TP_{ON} periods, and no energy is delivered during DOE1TP_{OFF} periods (e.g. a pulse-width modulation arrangement). In these embodiments, dose DOE2 can be delivered during DOE1TP_{OFF} periods of dose DOE1. In some embodiments, dose DOE2 comprises energy (e.g. an IEP dose) delivered in pulsed arrangement, such as when dose DOE2 comprises one or more periods of “on time” (DOE2TP_{ON}) separated by “off time” periods (DOE2TP_{OFF}), where energy is delivered during DOE2TP_{ON} periods, and no energy is delivered during DOE2TP_{OFF} periods (e.g. a pulse-width modulation arrangement). In these embodiments, dose DOE1 can be delivered during DOE2TP_{OFF} periods of dose DOE2. In some embodiments, doses DOE1 and DOE2 can each comprise energy delivered in a pulsed arrangement. In these embodiments, energy that is delivered during doses DOE1 and DOE2 can be delivered in an alternating arrangement, such as when energy delivery of dose DOE1 is delivered during a DOE2_{OFF} period of dose DOE2, and energy delivery of dose DOE2 can be delivered during a DOE1_{OFF} period of dose DOE1.

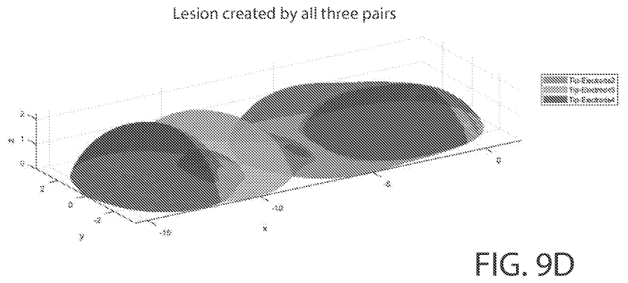

Referring now to FIG. 8, a side view of a distal portion of an energy delivery device is illustrated, consistent with the present inventive concepts. FIGS. 8A and 8B are two graphs of lesion depth versus electrode-pair during an electroporation study with the device of FIG. 8, consistent with the present inventive concepts. Referring additionally to FIGS. 9A-D, four graphs representing lesion volumes versus electrode-pair during the electroporation study with the device of FIG. 8 are illustrated, also consistent with the present inventive concepts. Applicant has conducted in-silico studies where an EDD 100, such as EDD 100 shown in FIG. 8, delivers a dose of energy comprising an IEP, as defined herein, to tissue (an IEP dose provided by EDC 300 as described herein in reference to FIG. 1). EDD 100 of FIG. 8 comprises four electrodes 130 (e.g. four electrode-based energy delivery elements 130) positioned in distal portion 102. EDD 100 comprises an electrode 130a positioned on the distal end of shaft 110, and three electrodes positioned more proximally on shaft 110, in a sequential, linear arrangement. Electrode 130b is positioned most proximate to tip electrode 130a, electrode 130c is positioned distal to electrode 130b. and electrode 130d is positioned distal to electrode 130c, all as shown in FIG. 8. Results of the in-silico studies indicated the shape and size of a lesion in tissue can be controlled by selecting a particular pair of electrodes 130, and having that pair deliver an IEP. FIGS. 8A-B illustrate testing from 3 pairs of electrodes 130, where pair “1-2” was electrode 130a and 130b, pair “1-3” was electrode 130a and 130c, and pair “1-4” was electrode 130a and 130d. In the simulation, the distal portion of EDD 100 was positioned approximately orthogonal (90°) to the tissue surface to receive the energy, electrode 130a had a length of 3.46 mm, the amplitude of the electric field was held constant. Tests were repeated for each of the 3 pairs at different voltages, 500V, 1000V, 1500V, and 2000V, as marked on the figure. FIG. 8A is a graph of lesion depth for each electrode 130 pair, while FIG. 8B is a graph of lesion surface area for each electrode 130 pair. FIGS. 9A-C are graphs of the volume of lesions created using pair 1-2, 1-3, and 1-4, respectively, where the distal portion of EDD 100 was placed on the tissue (e.g. at an angle of approximately 0°). FIG. 9D is a graph of the combined (superimposed) lesions of FIGS. 9A-C.

As shown by the studies, system 10 can be configured to switch between pairs of electrodes 130 receiving and/or delivering an IEP (without modifying amplitude), to modify lesion depth and/or to create lesions with desired geometric volumes. For example, as shown in FIG. 8A, a lesion created with an IEP of 1000V creates a deeper lesion when delivered by pair 1-3 than when delivered by pair 1-2. As shown in FIGS. 9A-D, a longer lesion (about 1.5 cm as shown) can be created by delivering IEP doses between multiple pairs of electrodes (e.g. without having to reposition the distal portion of EDD 100).

Referring now to FIGS. 10A-B, two anatomical sectional views of the distal portion of an energy delivery device contacting a tissue surface at different angles of orientation are illustrated, consistent with the present inventive concepts. As described herein, system 10, via EDD 100, can be configured to deliver one or more electric pulses between two or more electrodes 130 (e.g. one or more electrodes 130 configured to source current, and one or more electrodes 130 configured to sink current). The parameters of each of the pulses (e.g. voltage, current, frequency, pulse width, and the like) can be selected to create high voltage electric fields within tissue proximate the two or more electrodes, such as to perform “pulsed field ablation” to ablate the tissue. The energy is provided to EDD 100 by EDC 300, as described herein. The parameters of the electrical pulses can further be selected such that the resultant electric field causes reversible or irreversible electroporation of the tissue. The spatial extent (e.g. complete volume) of the tissue ablated and the effectiveness of the pulsed field ablation depends on the strength of the electric field at the target location (e.g. the target location selected for ablation to provide a therapeutic benefit to the patient as described herein). The strength of the electric field is related to the distance from the electrodes (e.g. electrodes 130 mounted to shaft 110 of EDD 100 as shown), where the field strength decreases exponentially with increasing distance from the electrodes. System 10 is configured to provide field strength at a sufficient level for all target tissue (e.g. all intended widths, lengths, and depths of target tissue) to be ablated.

System 10 (e.g. algorithm 335 described herein) can be configured to determine a lesion size parameter (e.g. a length, a width, a depth, and/or a volume of the lesion), L_{P}, where L_{P} is a function of: peak voltage of the pulsed field ablation (PFA), pulse V_{PEAK}; an angle of orientation α; and/or one or more electrophysical parameters EP_{P}. Angle α is the angle between the axis of distal portion 102 of EDD 100, and the plane of the tissue surface proximate distal portion 102 of EDD 100. In FIG. 10A, angle α is 90° (i.e. distal portion 102 of EDD 100 is orthogonal to the neighboring tissue surface), and in FIG. 10B, angle α is 0° (i.e. distal portion 102 of EDD 100 is parallel and in contact with the neighboring tissue surface). Parameter EP_{P} can include one, two, or more of: contact force, pulse amplitude, pulse duration, number of pulses, number of electrodes sourcing and/or sinking current; tissue temperature, and/or tissue impedance.

In some embodiments, EDD 100 comprises a sensor or other component configured to determine orientation angle α, such as to be used in a calculation to determine lesion size as described hereabove. For example, one or more sensors (e.g. sensing elements 158) of force maintenance assembly 150 (e.g. as described herein) can be configured to provide a signal from which angle α can be determined (e.g. by algorithm 335), such as when force maintenance assembly 150 comprises optical fibers, magnetic sensors, impedance measurement sensors, and/or other sensing elements configured to provide a signal related to angle α. Alternatively or additionally, angle α can be determined via signals provided by mapping and/or navigation sensors of system 10, such as when algorithm 335 performs impedance and/or magnetic based localization to determine angle α. Alternatively or additionally, system 10 can comprise an imaging device, not shown, but such as an imaging device selected from the group consisting of: intracardiac ultrasound image; x-ray; fluoroscope; magnetic resonance imager; computed tomography imager; visible light camera; infrared camera; and combinations of these. Algorithm 335 can utilize information provided by the imaging device to determine angle α.

In some embodiments, an electrical pulse is applied between tip electrode 130a and one or more neighboring electrodes 130 (e.g. electrodes 130b, 130c, and/or 130d shown). As it relates solely to angle α, the electric field strength in the tissue is at a minimum when angle α is at 90° as shown in FIG. 10A, and the field strength increases as angle α decreases from 90°, eventually reaching maximum when angle α is at 0° as shown in FIG. 10B. In other words, when one or more PFA pulses are delivered by EDD 100 to tissue, the spatial extent of the resultant lesion (e.g. the depth of the resultant lesion) increases as the electrodes 130 associated with each PFA pulse (e.g. the electrodes sourcing and/or sinking current) are moved closer to the tissue surface.

Referring now to FIGS. 11A-B, two user's views of a graphical user interface displaying information related to different angles of orientation of an energy delivery device are illustrated, consistent with the present inventive concepts. System 10, via user interface unit 320 of EDC 300, can include a graphical user interface, GUI 3200 shown. GUI 3200 can be configured to provide information related to orientation angle α described herein, such as is provided by orientation angle representation 3250 shown. GUI 3200 can display current (e.g. real time) position information related to EDD 100 (e.g. related to distal portion 102 of EDD 100). GUI 3200 can include: catheter representation 3210 representing the position of distal portion 102; and/or tissue representation 3220 representing the position of a tissue surface to be ablated (e.g. the tissue surface proximate distal portion 102); each as shown.

GUI 3200 can further comprise indicator graph 3230, which can provide field strength feedback to the operator, such as a graphical representation of an estimate of the field strength in the tissue proximate electrodes 130. The indicator graph 3230 can indicate the field strength of energy (e.g. PFA pulses currently being delivered, or the field strength that would be present once an operator initiates energy delivery). Information provided by indicator graph 3230, and other information provided by GUI 3200, can be used by an operator (e.g. in an iterative or other adjustable fashion), to create a lesion of a desired size (e.g. a desired length, width, and/or depth dimension). Indicator graph 3230 can include first marker 3231 and second marker 3232, each as shown. The relative position between the markers 3231 and 3232 can correlate to the field strength in tissue, such that marker 3231 approaches marker 3232 as the field strength increases (e.g. as shown in the transition from the orthogonal catheter orientation depicted in catheter representation 3210 of FIG. 11A transitioning to the catheter orientation depicted in catheter representation 3210 of FIG. 11B in which angle α is 5°). It should be understood that system 10 can provide various other forms of visual feedback to an operator regarding field strength and/or other ablation parameter of a current (e.g. real time) or future (to be delivered based on current conditions) pulsed field ablation energy delivery.

In some embodiments, GUI 3200 can be configured to provide information related to contact force, such as is provided by contact force representation 3260 shown, which includes force indicator 3261 representing the current contact force being applied (shown at the same level in each of FIGS. 11A and 11B). Contact force representation 3260 can further include threshold indicator 3262, which can indicate a required or recommended limit on contact force to be applied (e.g. an amount less than the maximum contact force available). The provided contact force information can provide an absolute measurement of force (e.g. a measurement expressed in grams or other metric indicating contact force), and/or a relative measurement (e.g. a percentage of a maximum amount of contact force). In some embodiments, GUI 3200 is further configured to change the distance between first marker 3231 and second marker 3232 as contact force changes, for example the distance can decrease (e.g. indicating an increase in electric field in tissue) when contact force increases. In some embodiments, the distance between the two markers is based on both angle α and contact force, where an operator can change either or both to change the electric field strength in the tissue. In some embodiments, GUI 3200 changes the distance between the two markers based on all or some of: angle α; contact force; and/or one or more electrophysical parameters EP_{P}.

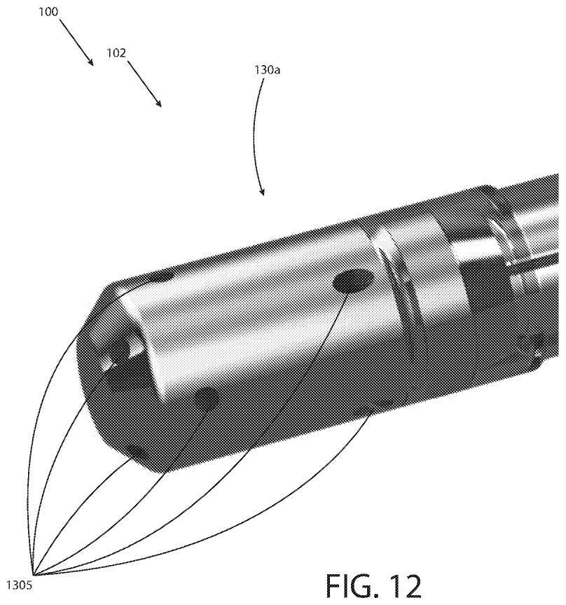

Referring now to FIG. 12, a perspective view of the distal portion of an energy delivery device including multiple ports for delivering irrigation fluid is illustrated, consistent with the present inventive concepts. EDD 100 of FIG. 12 includes electrode 130a shown on distal portion 102. The distal portion 102 of EDD 100 can include one or more ports, ports 1305 (six shown) for delivering irrigation fluid 70, such as one or more similar or dissimilar irrigation fluids 70 provided by fluid delivery module 370 of EDC 300 as described herein. Two or more ports 1305 can be spatially distributed in a desired pattern, such as a pattern covering a portion or the majority of distal portion 102 including electrodes 130. Two or more ports 1305 can be connected to independent lumens for delivery of different irrigation fluids 70 (e.g. different fluids 70a, 70b, and the like, such as fluids of dissimilar conductivity as described herein). Alternatively or additionally, two or more ports 1305 can be connected to a common lumen for delivery of the same irrigation fluid 70.

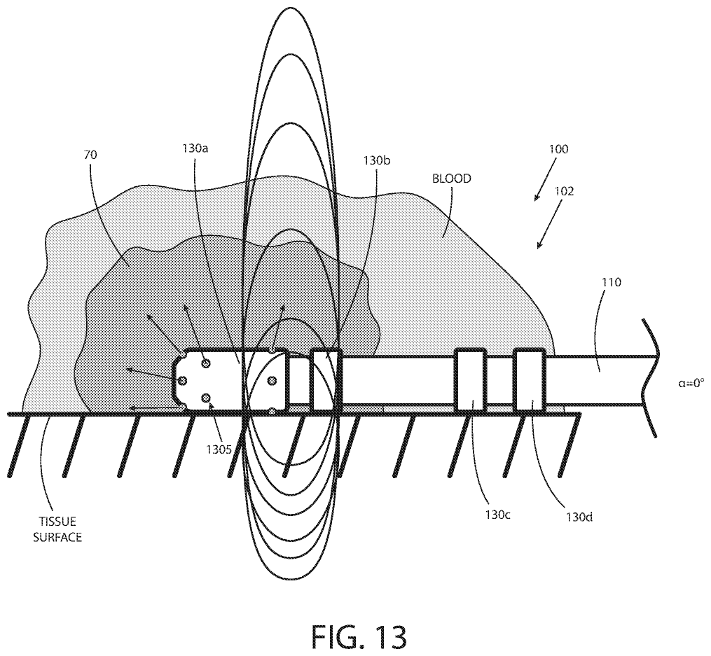

Referring additionally to FIG. 13, a side sectional anatomical view of the distal portion of an energy delivery device in contact with a tissue surface and delivering irrigation fluid is illustrated, consistent with the present inventive concepts. Distal portion 102 of EDD 100 is shown with an orientation angle α equal to 0°, such that each of electrodes 130a 130b, 130c and 130d are in contact with the tissue surface to be ablated. Irrigation fluid 70 is being delivered by ports 1305 (eight shown), and the delivered fluid 70 is surrounding electrodes 130a and 130b as shown (e.g. preventing blood, a relatively conductive substance, from surrounding those electrodes).

EDD 100 can be configured to deliver PFA pulses that create an electromagnetic field to ablate tissue (e.g. to cause reversible or irreversible electroporation of tissue as described herein). One or more irrigation fluids 70 can be delivered, via ports 1305, to influence the pulsed electric field. For example, a delivered electrical field will “bunch” when passing through conductive media, and it will “spread” when passing through more resistive media, due to the electric current following the path of least resistance (i.e. the path of highest conductivity). System 10 can be configured to deliver one or more irrigation fluids 70 of known conductance to the area surrounding electrodes 130, and actively “steer” the delivered current and therefore the produced electric field.

In some embodiments, distal portion 102 of EDD 100 comprises at least six ports 1305 (e.g. twelve ports 1305), such that at least two ports 1305 (e.g. four ports 1305) are facing forward (e.g. facing distally from the distal end of shaft 110), at least two ports 1305 (e.g. four ports 1305) are located at the distal end of tip electrode 130a, and at least two ports 1305 (e.g. four ports 1305) are located at the proximal end of tip electrode 130. System 10 can include irrigation fluid 70 having a different conductivity (e.g. lower conductivity) than the conductivity of blood. Prior to applying the pulsed electric field energy, EDD 100 can be oriented such that one or more ports 1305 are blocked via contact with tissue (e.g. blocked by the tissue surface of the left atrium or other chamber of the heart). Delivery of an irrigation fluid 70 via the remaining ports 1305 would have a steering effect on the desired electric field. For example, delivery of an irrigation fluid 70 having a lower conductivity than that of blood would concentrate the current delivered by electrode 130 into the contacting tissue, increasing the electric field into the tissue (e.g. as the fluid surrounding the electrode 130 delivering current is enveloped in the relatively low conductivity irrigation fluid 70).

Prior to, and/or during PFA pulse delivery, the angle of orientation α can be monitored as described herein (e.g. via one or more sensors of system 10 and/or by a separate imaging device), such as to provide feedback information relative to the electric field to be delivered and/or being delivered, where system 10 (e.g. algorithm 335, such as when algorithm 335 comprises an AI algorithm) can account for the increase in field strength (e.g. steering of the field) due to the delivery of the low conductance irrigation fluid 70.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which is defined in the accompanying claims.

1. A system for treating tissue of a patient, the system comprising:
an energy delivery console for providing a first dose of energy and a second dose of energy; and
an energy delivery device comprising a first delivery element configured to deliver the first dose of energy to target tissue, and a second delivery element configured to deliver the second dose of energy to the target tissue;
wherein the first dose of energy comprises a delivery of energy that reversibly alters the target tissue;
wherein the second dose of energy comprises a delivery of energy that irreversibly alters the target tissue; and
wherein the first dose of energy is delivered to enhance a therapy provided by the second dose of energy.

2.-54. (canceled)
